(19) United States
(12) Patent Application Publication
(10) Pub. No.: US 2026/0232590 A1
(43) Pub. Date: Aug. 13, 2026

(54) LIPID NANOPARTICLES FOR THE TREATMENT OF OCULAR DISEASES

(71) Applicant: UNIVERSITAT DE BARCELONA, Barcelona (ES)

(72) Inventors: Elena SÁNCHEZ LÓPEZ, Barcelona (ES); María Luisa GARCÍA LÓPEZ, Barcelona (ES); Lorena BONILLA VIDAL, Barcelona (ES); Marta ESPINA GARCÍA, Barcelona (ES)

(21) Appl. No.: 19/153,901

(22) PCT Filed: Feb. 6, 2024
(86) PCT No.: PCT/EP2024/052923

§ 371 Date: Aug. 5, 2025

(30) Foreign Application Priority Data
Feb. 6, 2023 (EP) 23382105.7

(51) Int. Cl. A61K 9/14; A61K 31/353; A61P 9/10; A61P 27/04; A61P 29/00; A61P 31/04; A61P 35/00

(52) U.S. Cl. CPC A61K 9/14; A61K 31/353; A61P 9/10; A61P 27/04; A61P 29/00; A61P 31/04; A61P 35/00

(57) ABSTRACT

The present invention relates to a composition comprising lipid nanoparticles comprising rosehip oil, at least one solid lipid, at least one surfactant, and optionally at least one active ingredient, as well as their use in a method of treating ocular diseases, such as for example dry eye disease.

### TECHNICAL FIELD

The present invention relates to a composition comprising lipid nanoparticles comprising rosehip oil, at least one solid lipid, at least one surfactant and optionally at least one active ingredient, as well as their use in a method of treating ocular diseases, such as for example dry eye disease.

### BACKGROUND OF THE INVENTION

Dry eye disease (DED) is a condition frequently encountered in ophthalmology practice worldwide. DED is a multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles. The prevalence of DED in the population ranges from 5-34% depending on the country [1]. Topical administration is the preferred route to treat DED because it is painless and easy to handle. Artificial tears in the form of eyedrops, gel or ointment are used to lubricate dry eyes maintaining moisture of the eye's surface and often constitute the first line of therapy. They instantly relieve symptoms by lowering osmolarity and diluting inflammatory markers. However, artificial tears have no anti-inflammatory properties and do not deal with the fundamental pathogenesis of the disease. Moreover, usual treatments for ocular inflammation comprise corticosteroids and non-steroidal anti-inflammatory drugs (NSAIDs), but its prolonged use involves severe side effects, such as increasing ocular pressure and cataract formation [2,3].

Plant oils have been used for a variety of purposes with their integration into foods, cosmetics, and pharmaceutical products. Specifically, the use of plant oils for topical skin applications as well as the therapeutic benefits of these plant oils according to their anti-inflammatory and antioxidant effects on the skin have been described. One of the plant oils used for such applications is rosehip oil.

Apigenin (APG) is a natural flavonoid that has been used in the form of plant extract for the treatment of several disorders and inflammatory conditions. Of all the flavonoids, APG is one of the most widely distributed in plants, and one of the most studied phenolics. APG is present in significant amount in vegetables, fruits, herbs and plant-based beverages. Based on preclinical and clinical data, it has been suggested that APG is a potent therapeutic agent to overcome diseases, such as inflammatory diseases, bacterial, viral, and parasitic infections, autoimmune disorders, diabetes, hypertension, hypercholesterolemia, and various types of cancers [4,5]. Several clinical trials have been carried out for the use of APG as a dietary supplement. In clinical trial NCT04114916, the reduction of cardiovascular risk in healthy subjects has been evaluated [6]. In clinical trial NCT01286324 APG, as a natural part of a chamomile extract, was studied as a dietary supplement for the treatment of chronic primary insomnia. It was concluded that the extract could provide modest benefits of daytime functioning and mixed benefits on sleep diary measures relative to placebo in adults with chronic primary insomnia [7]. Furthermore, APG itself is currently commercialized as a dietary supplement in Spain in the form of capsules containing 50 mg APG for improving prostate health, decrease glucose levels, and maintain the function of the nervous system [8,9].

Whilst commercialized eye drops containing chamomile extract for the enhancement of ocular discomfort, such as irritation, tired eyes, and itchiness are on the market [10,11], APG itself has to date not been suggested or approved as a therapy for ocular diseases, such as for example DED. This may partially be due to the disadvantages of APG for topical treatments caused among other by its low solubility and low bioavailability [12].

In the recent years new delivery systems, such as lipid nanoparticles, have been developed which provide multiple advantages.

As mentioned above topical administration is a preferred route to treat DED. The currently available remedies using artificial tears have no anti-inflammatory properties and do not deal with the fundamental pathogenesis of the disease. Available treatment options for ocular inflammation in general include corticosteroids and NSAIDs which after prolonged use can be the cause of severe side effects. Another drawback of currently available topical applications is the generally fast release of the active ingredient(s). Furthermore, topical applications into the eye require that the active ingredient is able to permeate through the corneal barrier to reach its destination.

To date, no lipid nanoparticles containing liquid lipids have been described that are suitable for topical use in the ocular system and that can provide benefits/treatment for ocular inflammatory diseases, such as DED. Furthermore, the simple and efficient treatment of ocular bacterial infections, neurodegenerative ocular pathologies and ocular tumours are areas that still require further advances.

The problem addressed by the present invention is to provide a topical ocular nanoparticulated lipid system comprising rose hip oil that provides biocompatibility, the capability to treat ocular disease or reverse their symptoms, treat and/or prevent ocular inflammation and the capacity to act as drug delivery carrier. A further problem addressed is the provision of a topical delivery system for APG that overcomes its disadvantages, such as its low solubility and bioavailability and that provides for sustained drug release and corneal permeability and thus improved pharmacokinetic and pharmacodynamic profiles of APG.

### BRIEF DESCRIPTION OF THE FIGURES

FIG. 1: Scheme of the evaluation of the fluorescein staining on the ocular surface. 9: maximum score; 0: minimum score.

FIG. 2: Scheme of the evaluation of Bengal rose staining on the ocular surface. 9: maximum score; 0: minimum score.

FIG. 3: Design of Experiments (DoE) surface response of Loaded Lipid Nanoparticles. (A) Concentration of APG (%) and glyceryl dibehenate+rosehip oil (%) influence on Zₐᵥ (nm). (B) Concentration of Polysorbate 80(%) and glyceryl dibehenate+rosehip oil (%) influence on PDI. (C) Concentration of Polysorbate 80(%) and glyceryl dibehenate+rosehip oil (%) influence on ZP (mV). (D) Concentration of APG (%) and glyceryl dibehenate+rosehip oil (%) influence on EE (%).

FIG. 4: Physicochemical characterization of optimized Loaded Lipid Nanoparticles, Lipid Nanoparticles and apigenin. (A) Transmission electron microscopy (TEM) image of loaded lipid nanoparticles (scale bar 100 nm). (B) Differential scanning calorimetry (DSC) curves. (C) X-ray diffraction (XRD) patterns. (D) Fourier-transformed infrared (FTIR) analysis.

FIG. 5: Backscattering profiles of Loaded Lipid Nanoparticles stored at: (A) 4° C. and, (B) 25° C.

FIG. 6: Biopharmaceutical behavior. In vitro APG release from Loaded Lipid nanoparticles against free-APG (adjusted to two phase decay and plateau followed by one phase decay respectively) and pharmacokinetic parameters Loaded Lipid Nanoparticles applied to a one-phase association.

FIG. 7: Results of the Schirmer test on New Zealand rabbits after dry eye disease induction with statistically significant differences (* p<0.05, ** p<0.01, *** p<0.001; **** p<0.0001).

FIG. 8: Fluorescein staining of the ocular surface of New Zealand rabbits after dry eye disease induction. Results of the fluorescein staining with statistically significant differences (* p<0.05, ** p<0.01, and *** p<0.001) of: Dry eye, Lipid Nanoparticles, Loaded Lipid Nanoparticles, Free APG, Commercial solution.

FIG. 9: Bengal rose staining of the ocular surface of New Zealand rabbits after dry eye disease induction. Results of the Bengal rose staining with statistically significant (* p<0.05) of: Dry eye, Lipid Nanoparticles, Loaded Lipid Nanoparticles, Free APG, Commercial solution.

FIG. 10: Effect of free APG, Loaded Lipid Nanoparticles and Lipid Nanoparticles on the viability of HCE-2 cells at 5, 15, or 30 minutes. The 100% cell viability corresponds with the average of MTT reduction values of untreated cells. A) Cell viability of free APG; B) cell viability of Loaded Lipid Nanoparticles; C) cell viability of Lipid Nanoparticles, in which the used concentration was the same than in the Loaded Lipid Nanoparticles

FIG. 11: Effect of free APG, Loaded Lipid Nanoparticles and Lipid Nanoparticles on the viability of UM 92.1 cancer cells. Control (CTRL) column corresponds to the 100% cell viability average of MTT reduction values of untreated cells. A) Cell viability of free APG; B) cell viability of Loaded Lipid Nanoparticles; C) cell viability of Lipid Nanoparticles. D) Cell viability comparison between free APG, Loaded Lipid Nanoparticles and Lipid Nanoparticles against UM 92.1 cell line. Values are expressed as mean±SD; *p<0.05, ** p<0.01, ***p<0.005, and ****p<0.001 significantly lower than the control cells.

FIG. 12: Cellular uptake of Loaded Lipid Nanoparticles in HCE-2 at different incubation times (5, 15, or 30 minutes). White arrows highlight the samples localization.

FIG. 13: Antiangiogenic capacity of free APG-Loaded Lipid Nanoparticles, and Lipid Nanoparticles. Values are expressed as mean±SD; *p<0.05, ** p<0.01, ***p<0.005, and ****p<0.001 significantly lower.

FIG. 14: Antibacterial activity of free APG-Loaded Lipid Nanoparticles, and Lipid Nanoparticles. Values are expressed as mean±SD; ***p<0.005.

FIG. 15: Comparison of ocular anti-inflammatory efficacy of free APG, Loaded Lipid Nanoparticles, and Lipid Nanoparticles. (A) Inflammation treatment, (B) inflammation prevention. Values are expressed as mean±SD; *p<0.05, **p<0.01, ***p<0.005, and ****p<0.001 significantly lower than the inflammatory effect induced by SA; $p<0.05 and $$p<0.05 significantly lower effect of Loaded Lipid Nanoparticles than the inflammatory effect induced by Lipid Nanoparticles; &&&&p<0.0001 significantly lower effect of Loaded Lipid Nanoparticles than the corresponding free drug; #p<0.05 and ##p<0.01 significantly lower effect of Lipid Nanoparticles than the corresponding free drug.

FIG. 16: Ocular biodistribution images in New Zealand rabbits. Anterior (A) and posterior (P) segments are indicated.

### SUMMARY OF THE INVENTION

The inventors have developed lipid nanoparticles based on rosehip oil with intrinsic activity that are suitable for the applications mentioned above and that provide biocompatibility, capability to reverse disease symptoms as well as anti-inflammatory, antibacterial, antitumoral- and antiangiogenic efficacy. In addition, these lipid nanoparticles present good long-term stability, easy production at large scale, and increased kinetic stability. Thus, these lipid nanoparticles represent a topical delivery system with a high performance and, themselves are efficient in the treatment of ocular inflammatory diseases, while at the same time reducing the side effects that can occur with drugs used in topical eye treatments.

In addition, the inventors have provided a topical delivery system that is especially suitable for the application of APG overcoming its low solubility and low bioavailability, and that furthermore provides for sustained drug release and corneal permeability which is essential for improving the pharmacokinetic and pharmacodynamic profile of APG and allows simple and effective treatment with low side effects

Accordingly, one aspect the present invention relates to a composition comprising lipid nanoparticles, said lipid nanoparticles comprising
a) rosehip oil;
b) at least one solid lipid;
c) at least one surfactant;
d) optionally, at least one active ingredient;
   wherein the composition is suitable for ocular topical administration.

In one embodiment of the composition of the present invention the solid lipid is selected from monoglycerides, diglycerides, triglycerides, cholesterols, steroids, fatty alcohols, glycerol esters glyceryl tridecanoate, glycerol trilaurate, glyceryl trimyristate, glyceryl tripalmitate, glyceryl tristearate, hydrogenated coco-glycerides, hard fat types, mixtures of triglycerides and/or diglycerides and/or monoglycerides and/or glycerol, acyl glycerols, glyceryl monostearate, glyceryl distearate, glyceryl monooleate, glyceryl dibehenate, glyceryl palmitostearate, waxes, cetyl palmitate, fatty acids, stearic acid, palmitic acid, decanoic acid, behenic acid, glycerol stearate citrate, polyethylene glycol monostearate, cyclodextrin para-acyl-calix-arenes, or mixtures thereof.

In a preferred embodiment said solid lipid is glyceryl dibehenate.

In one embodiment the at least one surfactant is selected from a cationic surfactant, a non-cationic surfactant, or a combination thereof.

In one embodiment of the composition comprising lipid nanoparticles of present invention the cationic surfactant is selected from the group consisting of dimethyl-dioctadecyl-ammonium bromide (DDAB), dioleoyl phosphatidylethanolamine, 1,2-distearyloxy-N,N-dimethyl-3-aminopropane, 1,2 dioleyloxy-N,N-dimethyl-3-aminopropane, 1,2-dilinoleyloxy-N,N-dimethyl-3-aminopropane, 1,2-dilinolenyloxy-N,N-dimethyl-3-aminopropane, cetyltrimethylammonium bromide, 3ß-[N(N′,N′-dimethylaminoethane)-carbamoyl]cholesterol, 1,2-dioleoyl-3-trimethylammonium-propane, 1,2-dimyristoyl-3-trimethylammonium-propane, 1,2-stearoyl-3-trimethylammonium-propane, N-(4-carboxybenzyl)-N,N-dimethyl-2,3-bis(oleoyl-oxy)propan-1-aminium, 1-Palmitoyl-2-oleoyl-sn-glycero-3-phosphoethanolamine, N,N-di-(β-stearoylethyl)-N,N-dimethyl-ammonium chloride, benzalkonium chloride, cetylpyridinium chloride, cetrimide, N-[1-(2,3-dioleoyloxy)propyl]-N,N,N-trimethylammonium chloride, 1,2-dilineoyl-3-dimethylammonium-propane, 1,2-dilinoleyloxy-3-N,N-dimethylaminopropane, 1,2-dilinoleyloxy-keto-N,N-dimethyl-3-aminopropane, 1,2-dilinoleyl-4-(2-dimethylaminoethyl)-[1,3]-dioxolane(3-o-[2″-(meth oxypolyethyleneglycol 2000) succinoyl]-1,2-dimyristoyl-sn-glycol, R-3-[(ω-methoxy-poly(ethyleneglycol)2000)carbamoyl]-1,2-dimyristyloxlpropyl-3-amine, cetyl-trimethylammonium bromide, octadecylamine, 1-oleoyl-rac-glycerol, octadecyl quaternized carboxymethyl chitosan, hexadecyl trimethyl ammonium bromide.

In a preferred embodiment of the composition of present invention the cationic surfactant is dimethyldioactadecylammonium bromide (DDAB).

In a further embodiment of the composition of present invention the non-cationic surfactant is selected from the group consisting of polysorbate 80, soya lecithin, sodium dodecyl sulphate, polysorbate 20, polysorbate 40, polysorbate 60, PEG-30 glyceryl stearate, cholic acid, phosphatidyl choline, phospholipids with phosphatidylcholine, egg lecithin, poloxamer 188, poloxamer 407, poloxamer 184, poloxamer 338, poloxamine 908, tyloxopol, taurocholate sodium salt, taurodeoxycholicacid sodium salt, sodium glycocholate, sodium oleate, cholesteryl hemisuccinate, butanol, sodium cholate, nonionic polyoxyethylene, non-ionic amphiphilic surfactant with an alkyl moiety and an ethylene oxide chain, palmitic acid, stearic acid, mixtures of palmitic and stearic acid, lecithin, polyglycerol 6-distearate, caprylyl/capryl glucoside, coco-glucoside, sucrose palmitate, sucrose stearate, sucrose distearate, tyloxapol, lecithin, cetylpyridinium chloride, sorbitan laurate, polyethylene glycol ether of cetyl or stearyl alcohol, castor oil polyoxyethylene ether, macrogolglycerol ricinoleate, dioctyl sodium sulfosuccinate, monooctylphosphoric acid sodium, hexadecyl trimethyl ammonium bromide, polyvinyl alcohol, polyoxyethylene (40) stearate, polyethylene glycol-polypropylene glycol-polyethylene glycol triblock copolymer, olyoxyethylene nonylphenyl ether, hexadecyltrimethylammonium bromide, sodium dodecyl sulfate, sodium cholate, stearate sodium hydrolysed polyvinyl alcohol 9000-10000 MW, dioctyl sulfosuccinate, taurocholate, 4-dodecylbenzenesulfonic acid, long chain carboxylic acid, alkyldiphenyloxide disulfonate, or mixtures thereof.

In a preferred embodiment the non-cationic surfactant is polysorbate 80.

In one embodiment the at least one active ingredient, preferably apigenin, is encapsulated in the lipid nanoparticles.

In one embodiment the at least one active ingredient is apigenin (APG).

In a further embodiment said lipid nanoparticles further comprise a coating, preferably wherein said coating comprises hyaluronic acid.

In one preferred embodiment of the composition of present invention said lipid nanoparticles comprise a. 0.01-30% (w/v) rosehip oil;
b. 1.0-50% (w/v) solid lipid, preferably glyceryl dibehenate;
c. 0.001-10% (w/v) cationic surfactant, preferably DDAB;
d. optionally 0.00-10% (w/v) non-cationic surfactant, preferably polysorbate 80;
e. optionally 0.00-10% (w/v) active ingredient, preferably apigenin.

In one preferred embodiment of the composition of present invention said lipid nanoparticles comprise
a. 2.5-12.5% (w/v) rosehip oil;
b. 1.0-30% (w/v) solid lipid, preferably glyceryl dibehenate;
c. 0.025-0.5% (w/v) cationic surfactant, preferably DDAB;
d. 1.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise 1.0-6.5% (w/v) solid lipid and 0.03-0.4% (w/v) cationic surfactant.

In one preferred embodiment of the composition of present invention said lipid nanoparticles comprise
a. 2.5-10% (w/v) rosehip oil;
b. 1.0-6.5% (w/v) solid lipid, preferably glyceryl dibehenate;
c. 0.03-0.4% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 3.0% (w/v) rosehip oil;
b. 4.5% (w/v) glyceryl dibehenate,
c. 0.05% (w/v) DDAB,
d. 3.5% (w/v) polysorbate 80,
e. optionally 0.1% (w/v) apigenin.

In one embodiment the lipid nanoparticles further comprise from 0.00001%-0.001% (w/v), preferably 0.0001% (w/v) hyaluronic acid, more preferably wherein said hyaluronic acid coats the lipid nanoparticles.

In another aspect the present invention relates to a composition comprising lipid nanoparticles as described herein for use in the treatment, amelioration or prevention of ocular diseases, such as ocular inflammation, glaucoma, ocular tumors, bacterial and viral infections of the eye, age-related macular degeneration, cataracts, diabetic retinopathy, or dry eye disease (DED).

In one embodiment the composition is administered topically into the eye.

### DETAILED DESCRIPTION OF THE INVENTION

### Definitions

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, or 10%. Accordingly, the term “about” may mean the indicated value ±5% of its value, preferably the indicated value ±2% of its value, most preferably the term “about” means exactly the indicated value (±0%).

The terms “comprise” and “comprising” are used in the inclusive, open sense, meaning that additional elements may be included. The term “comprises” also encompasses and may be used interchangeably with the terms “consists of” and “consists essentially of”.

### DETAILED DESCRIPTION

Dry eye disease (DED) is a condition frequently encountered in ophthalmology practice worldwide. Topical administration is a preferred route to treat DED, but currently available artificial tears have no anti-inflammatory properties and do not deal with the fundamental pathogenesis of the disease. Usual treatments for ocular inflammation include corticosteroids and non-steroidal anti-inflammatory drugs (NSAIDs) which after prolonged use led to severe side effects. Another drawback of topical applications is the generally fast release of the active ingredient(s). Furthermore, for topical applications into the eye, it needs to be assured that the active ingredient can permeate through the corneal barrier to reach its destination.

The inventors have therefore set out to develop a drug delivery system that can overcome these drawbacks and that provides biocompatibility, anti-inflammatory efficacy as well as capability to reverse ocular disease symptoms, such as DED. A further aim of the invention is to provide an ocular topical delivery system for APG that overcomes its low solubility and bioavailability and that provides for sustained drug release as well as corneal permeability thus improving the pharmacokinetic and pharmacodynamic profile of APG.

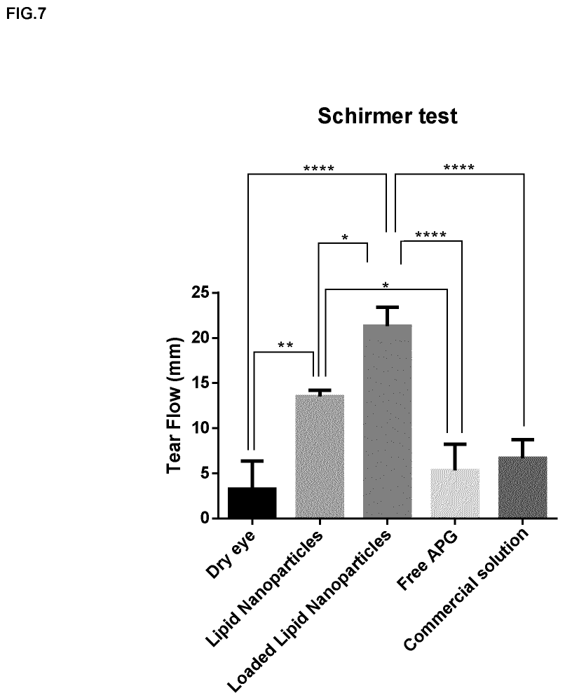

To achieve this the inventors have developed lipid nanoparticles comprising rosehip oil extracted from Rosa canina seeds as a liquid lipid having antioxidant, anti-inflammatory and regenerative properties. Rosehip oil contains essential fatty acids, tocopherols, sterols and phenolics with functional properties. The inventors could show that the lipid nanoparticles according to present invention comprising rosehip oil and no further active ingredient, herein named “lipid nanoparticles”, already provide for an improved tear flow, could restore the corneal surface and were able to restore the tear film (see Example 5, FIGS. 7-9).

In one aspect the present invention therefore relates to a composition comprising lipid nanoparticles, said lipid nanoparticles comprising
a. rosehip oil;
b. at least one solid lipid;
c. at least one surfactant
d. optionally, at least one active ingredient.
   wherein the composition is suitable for ocular topical administration.

In one embodiment of present invention the at least one surfactant is selected from a cationic surfactant, a non-cationic surfactant, or a combination thereof.

In one preferred embodiment of present invention the at least one surfactant is a cationic surfactant. In a more preferred embodiment, the composition further comprises a non-cationic surfactant.

In one preferred embodiment the composition of present invention is suitable for ocular topical administration. The ocular administration can be via direct administration into the eye, such as for example onto the cornea or into the conjunctival sac. In a preferred embodiment the composition is administered as an eye drop. It is to be understood that such topical administration differs from systemic ways of administration, such as oral or intravenous administration, and also from the administration via vitreous or periocular injection.

In one embodiment the lipid nanoparticles comprise at least one further liquid lipid other than rose hip oil. In one embodiment this further liquid lipid has anti-inflammatory properties. In one embodiment this liquid lipid with anti-inflammatory properties can be selected from the group consisting of tea tree oil, lavender oil, linoleic acid, stearic acid, palmitic acid, castor oil, safflower oil, melon seed oil, salicornia oil, evening primrose oil, poppyseed oil, grape seed oil, prickly pear oil, artichoke oil, hemp oil, wheat germ oil, cottonseed oil, corn oil, walnut oil, soybean oil, sesame oil, rice bran oil, argan oil, pistachio oil, peach oil, almond oil, canola oil, avocado oil, flaxseed oil, sunflower oil, peanut oil, palm oil, olive oil, macadamia oil, coconut oil, rosemary oil, lavender oil, Origanum vulgare oil, thyme oil, mint oil, eucalyptus oil, ginger oil, Cuminum cyminum I. oil, turmeric oil, clove oil, oleic acid, oregano oil, rose oil, fennel oil, bergamot oil, chamomile oil, helichrysum oil, patchouli oil, frankincense oil, copaiba oil, peppermint oil, black pepper oil, sweet marjoram oil, basil oil, clove oil, clary sage oil, lemongrass oil, geranium oil, wintergreen oil, cannabis oil, cannabidiol, spruce oil, niaouli oil, cardamom oil, pine tree oil, fir tree oil, juniper tree oil, verbena oil, marjoram oil, katafray oil, bitter orange oil, hypericum oil, arnica oil, coriander oil, mustard oil, perilla seed oil, Centella asiatica oil, calendula oil, laurel oil, camphor oil, cinnamon oil, oatmeal oil, docosahexaenoic acid, eicosapentaenoic acid, dandelion oil, krill oil, electrophorus electricus oil, potamotrygon motoro oil, boa constrictor oil, chelonoidis denticulate oil, Melanosuchus niger oil, Inia geoffrensis oil, horse oil, anchovy oil, prunus seed oil, Tropidurus hispidus oil, emu oil, maqian fruits essential oil, fructus alpinia oil, Cinnamomum cassia essential oil, Angelica sinensis oil, Gynura procumbens oil, spirulina oil, citrus limetta oil, citrus aurantium oil, Atractylodes macrocephala oil, Artemisia argyi oil, Gynura procumbens oil, Acorus gramineus and oil, algal oil, fish oil, Zanthoxylum coreanum nakai oil, cod liver oil, Perna canaliculus oil, chia seed oil, or combinations thereof.

The above listed liquid lipids are known to contain anti-inflammatory properties and are thus suitable for use in the compositions of present invention. Anti-inflammatory properties herein refer to the ability of the liquid lipid to reduce inflammation and/or redness, and/or pain and/or swelling of a tissue, specifically of the eye.

The lipid nanoparticles of present invention can comprise from 0.01-30.0% (w/v), from 0.1-30.0% (w/v), from 0.5-25.0% (w/v), from 1.0-20.0% (w/v), from 1.5-15.0% (w/v), from 2.0-14.0% (w/v), from 2.5-12.5% (w/v), from 2.5-10.0% (w/v), from 2.5-9.0% (w/v), from 2.5-8.0% (w/v), from 2.5-7.0% (w/v), from 2.7-6.0% (w/v), from 2.8-5.0% (w/v), from 2.9-4.0% (w/v) liquid lipid with anti-inflammatory properties, such as rosehip oil. Preferred is an amount of from 2.5-12.5% (w/v), more preferred an amount from 2.9-4.0% (w/v), most preferred 3.0% (w/v). The preferred liquid lipid is rosehip oil.

In one embodiment the lipid nanoparticles can comprise a combination of liquid lipids with anti-inflammatory properties as listed above.

In one embodiment of the composition of the present invention the solid lipid is selected from monoglycerides, diglycerides, triglycerides, cholesterols, steroids, fatty alcohols, glycerol esters glyceryl tridecanoate, glycerol trilaurate, glyceryl trimyristate, glyceryl tripalmitate, glyceryl tristearate, hydrogenated coco-glycerides, hard fat types, mixtures of triglycerides and/or diglycerides and/or monoglycerides and/or glycerol, acyl glycerols, glyceryl monostearate, glyceryl distearate, glyceryl monooleate, glyceryl dibehenate, glyceryl palmitostearate, waxes, cetyl palmitate, fatty acids, stearic acid, palmitic acid, decanoic acid, behenic acid, glycerol stearate citrate, polyethylene glycol monostearate, cyclodextrin para-acyl-calix-arenes, or mixtures thereof.

The solid lipid can be present in the lipid nanoparticles of present invention from 1.0-50% (w/v), from 1.0-40% (w/v), from 1.0-30% (w/v), from 1.0-20% (w/v), from 1.0-15% (w/v), from 1.0-10% (w/v), from 1.5-9.5% (w/v), from 2.0-9.0% (w/v), from 2.5-8.5% (w/v), from 3.0-8.0% (w/v), from 3.2-7.5% (w/v), from 3.5-7.0% (w/v), from 3.7-6.5% (w/v), from 1.0-6.5% (w/v), from 1.5-6.5% (w/v), from 2.0-6.5% (w/v), from 2.5-6.5% (w/v), from 3.0-6.5% (w/v), from 3.5-6.5% (w/v), from 4.0-6.5% (w/v), from 1.0-6.0% (w/v), from 1.5-6.0% (w/v), from 2.0-6.0% (w/v), from 2.5-6.0% (w/v), from 3.0-6.0% (w/v), from 3.5-6.0% (w/v), from 4.0-6.0% (w/v), from 1.0-5.5% (w/v), from 1.5-5.5% (w/v), from 2.0-5.5% (w/v), from 2.5-5.5% (w/v), from 3.0-5.5% (w/v), from 3.5-5.5% (w/v), from 4.0-5.5% (w/v), from 1.0-6.0% (w/v), from 4.1-5.5% (w/v), from 4.2-5.0% (w/v), from 4.3-4.9% (w/v), from 4.4-4.8% (w/v).

The lipid nanoparticles comprise at least one surfactant. The surfactant can be a cationic or a non-cationic surfactant. Preferred is that the lipid nanoparticles comprise at least one cationic surfactant. The cationic surfactant provides an increased bioavailability of the formulation when administered onto the ocular mucosa. Cationic surfactants promote electrostatic interactions between the surface of the cationic particles and the anionic ocular mucosa, with a considerable improvement of the drug residence time [13].

In one embodiment of the composition of present invention the cationic surfactant is selected from the group consisting of dimethyldioctadecylammonium bromide (DDAB), dioleoyl phosphatidylethanolamine, 1,2-distearyloxy-N,N-dimethyl-3-aminopropane, 1,2 dioleyl-oxy-N,N-dimethyl-3-aminopropane, 1,2-dilinoleyloxy-N,N-dimethyl-3-aminopropane, 1,2-dilinolen-yloxy-N,N-dimethyl-3-aminopropane, cetyltri-methylammonium bromide, 3ß-[N(N′,N′-dimethylaminoethane)-carbamoyl]cholesterol, 1,2-dioleoyl-3-trimethylammonium-propane, 1,2-dimyristoyl-3-trimethylammonium-propane, 1,2-stearoyl-3-tri-methylammonium-propane, N-(4-carboxybenzyl)-N,N-dimethyl-2,3-bis(oleoyloxy) propan-1-aminium, 1-Palmitoyl-2-oleoyl-sn-glycero-3-phosphoethanolamine, N,N-di-(β-stearoylethyl)-N,N-dimethyl-ammonium chloride, benzalkonium chloride, cetylpyridinium chloride, cetrimide, N-[1-(2,3-dioleoyloxy)propyl]-N,N,N-trimethylammonium chloride, 1,2-dilineoyl-3-dimethylammonium-propane, 1,2-dilinoleyloxy-3-N,N-dimethylaminopropane, 1,2-dilinoleyloxy-keto-N,N-dimethyl-3-aminopropane, 1,2-dilinoleyl-4-(2-dimethylaminoethyl)-[1,3]-dioxolane(3-o-[2″-(meth oxypolyethyleneglycol 2000) succinoyl]-1,2-dimyristoyl-sn-glycol, R-3-[(ω-methoxy-poly(ethyleneglycol)2000) carbamoyl]-1,2-dimyristyloxlpropyl-3-amine, cetyltri-methylammonium bromide, octadecylamine, 1-oleoyl-rac-glycerol, octadecyl quaternized carboxymethyl chitosan, hexadecyl trimethyl ammonium bromide.

A preferred cationic surfactant according to present invention is dimethyl-dioctadecyl-ammonium bromide (DDAB). DDAB is a commercially available double chain cationic surfactant that is for example used in the prior art for preparation of lipid bilayer-protected gold nanoparticles (AuNPs) or for delivery systems into mammalian cells, such as RNAi delivery.

The lipid nanoparticles of present invention can comprise from 0.001-10% (w/v), from 0.0015-5% (w/v), from 0.010-4% (w/v), from 0.015-2% (w/v), from 0.020-1% (w/v), from 0.021-0.9% (w/v), from 0.022-0.8% (w/v), from 0.023-0.7% (w/v), from 0.024-0.6% (w/v), from 0.025-0.5% (w/v), from 0.030-0.4% (w/v), from 0.035-0.3% (w/v), from 0.040-0.2% (w/v), from 0.045-0.1% (w/v) of a cationic surfactant.

Cationic surfactants can cause irritation and even toxicity at higher concentrations. However, a certain amount of cationic surfactant is beneficial for providing the desired properties to the nanoparticles of present invention. In previous studies for ocular delivery DDAB had not shown any toxicity at a concentration of 0.5%. The inventors have now found that an amount of less than 0.5% (w/v) and even as little as 0.05% (w/v) of cationic surfactant, specifically DDAB, was sufficient for providing the composition with the desired properties. As such, in one preferred embodiment the lipid nanoparticles of present invention comprise less than 0.5% (w/v), more preferred 0.05% (w/v) of DDAB.

In one embodiment the lipid nanoparticles further comprise at least one further surfactant that is not a cationic surfactant, herein referred to also as “non-cationic surfactant”. Surfactant type and concentration play an important role in designing lipid nanoparticles. Lipid nanoparticles are stabilized by different types of surfactants which are efficiently adsorbed onto particles' surfaces reducing the interfacial tension. Either a sole surfactant or a mixture of hydrophilic and lipophilic surfactants can be used for the preparation. A blend can provide improved physical stability and functional properties to the lipid nanoparticles.

In one embodiment the lipid nanoparticles comprise only a non-cationic surfactant.

In one embodiment of the composition of present invention the non-cationic surfactant is selected from the group consisting of polysorbate 80, soya lecithin, sodium dodecyl sulphate, polysorbate 20, polysorbate 40, polysorbate 60, PEG-30 glyceryl stearate, cholic acid, phosphatidyl choline, phospholipids with phosphatidycholine, egg lecithin, poloxamer 188, poloxamer 407, poloxamer 184, poloxamer 338, poloxamine 908, tyloxopol, taurocholate sodium salt, taurodeoxycholicacid sodium salt, sodium glycocholate, sodium oleate, cholesteryl hemisuccinate, butanol, sodium cholate, nonionic polyoxyethylene, non-ionic amphiphilic surfactant with an alkyl moiety and an ethylene oxide chain, palmitic acid, stearic acid, mixtures of palmitic and stearic acid, lecithin, polyglycerol 6-distearate, caprylyl/capryl glucoside, coco-glucoside, sucrose palmitate, sucrose stearate, sucrose distearate, tyloxapol, lecithin, cetylpyridinium chloride, sorbitan laurate, polyethylene glycol ether of cetyl or stearyl alcohol, castor oil polyoxyethylene ether, macrogolglycerol ricinoleate, dioctyl sodium sulfosuccinate, monooctylphosphoric acid sodium, hexadecyl trimethyl ammonium bromide, polyvinyl alcohol, polyoxyethylene (40) stearate, polyethylene glycol-polypropylene glycol-polyethylene glycol triblock copolymer, olyoxyethylene nonylphenyl ether, hexadecyltrimethylammonium bromide, sodium dodecyl sulfate, sodium cholate, stearate sodium hydrolysed polyvinyl alcohol 9000-10000 MW, dioctyl sulfosuccinate, taurocholate, 4-dodecylbenzenesulfonic acid, long chain carboxylic acid, alkyldiphenyloxide disulfonate, or mixtures thereof.

The concentration of the non-cationic surfactant particularly influences the particle size of the lipid nanoparticles. Generally, the higher the non-cationic surfactant concentration, the smaller the particle sizes will be. The non-cationic surfactant can be present in the lipid nanoparticles from 0.01-10% (w/v), from 0.1-9.5% (w/v), from 0.2-9.0% (w/v), from 0.3-8.5% (w/v), from 0.4-8.0% (w/v), from 0.5-7.5% (w/v), from 0.6-7.0% (w/v), from 0.7-6.5% (w/v), from 0.8-6.0% (w/v), from 0.9-5.5% (w/v), from 1.0-5.0% (w/v), from 1.5-4.5% (w/v), from 2.0-4.0% (w/v), from 2.5-3.5% (w/v). The non-cationic surfactant can be present in the lipid nanoparticles from 3.0% (w/v) −10% (w/v), from 3.0% (w/v)-9.5% (w/v), from 3.0% (w/v)-9.0% (w/v), from 3.0% (w/v)-8.5% (w/v), from 3.0% (w/v)-8.0% (w/v), from 3.0% (w/v)-7.5% (w/v), from 3.0% (w/v)-7.0% (w/v), from 3.0% (w/v)-6.5% (w/v), from 3.0% (w/v)-6.0% (w/v), from 3.0% (w/v)-5.5% (w/v), from 3.0% (w/v)-5.0% (w/v), from 3.0% (w/v)-4.5% (w/v), from 3.0% (w/v)-4.0% (w/v). In one preferred embodiment the non-cationic surfactant can be present in the lipid nanoparticles at 3.5% (w/v). For clarity, the ranges disclosed refer to the amount of non-cationic surfactant in the lipid nanoparticles, thus not including the amount of cationic surfactant. The amount of cationic surfactant is specified further above.

The lipid nanoparticles of present invention can further comprise at least one active ingredient. In one embodiment the at least one active ingredient is selected from the group consisting of flavonoids, resveratrol (3,5,4′-trihydroxy-trans-stilbene) and curcumin. Preferred active ingredients are flavonoids due to their antioxidant and anti-inflammatory properties.

A preferred active ingredient of present invention is apigenin. APG is a natural flavonoid that is contained in various plant extracts, among others chamomile extracts, and has been used as plant extract for the treatment of several disorders and inflammatory conditions. Based on preclinical and clinical data, it has been suggested that APG is a potent therapeutic agent to overcome diseases, such as inflammatory diseases, bacterial, viral, and parasitic infections, autoimmune disorders, diabetes, hypertension, hypercholesterolemia, and various types of cancers [4,5]. Furthermore, APG itself is currently commercialized as a dietary supplement in Spain in the form of capsules containing 50 mg APG for improving prostate health, decrease glucose levels, and maintain the function of the nervous system [8,9].

Whilst commercialized eye drops containing chamomile extract for the enhancement of ocular discomfort, such as irritation, tired eyes, and itchiness are on the market [10,11], APG itself has to date not been suggested or approved as a therapy for ocular diseases, such as for example DED. This may partially be due to the disadvantages of APG for topical treatments caused among other by its low solubility and low bioavailability [12]. The same problem occurs with other flavonoids. To overcome these disadvantages the inventors have encapsulated APG into the lipid nanoparticles as described above. APG encapsulation into biocompatible and biodegradable lipid nanoparticles has been carried out to overcome its compromised stability and increase therapeutic activity and half-life on the ocular surface, granting its prolonged release. The inventors have shown that apigenin has very good compatibility with the nanoparticles of present invention containing rose hip oil as described in further detail here below.

In a further embodiment said lipid nanoparticles further comprise a coating. The coating can comprise carbomers, such as CMC (carboxymethyl cellulose), chitosan or hyaluronic acid. In a preferred embodiment said coating comprises hyaluronic acid. Hyaluronic acid (HA) is one of the most utilised viscosity-building macromolecules in ocular delivery devices. This anionic polysaccharide with ocular mucomimetic properties exhibits the capacity of prolonging the precorneal residence time and reducing surface desiccation [14]. In addition, hyaluronic acid can act as a lubricant and provide for an additional moisturizing effect.

This nanosystem has been proven to be physically stable with a prolonged APG release as well as high corneal permeability, thus improving biopharmaceutical APG behavior. In addition, in vitro and in vivo tests corroborate that the developed formulation is biocompatible without any sign of ocular irritation. As already described above, Lipid Nanoparticles showed an ability to revert DED symptoms due to its composition. When the lipid nanoparticles were loaded with APG (“Loaded Lipid Nanoparticles”), tear secretion improved due to the therapeutic properties of APG. In addition, a complementary anti-inflammatory effect of such Loaded Lipid Nanoparticles is also confirmed (see Example 5, FIGS. 7-9). Hence, Loaded Lipid Nanoparticles constitute a suitable system for the treatment, amelioration and prevention of ocular inflammatory diseases, such as DED.

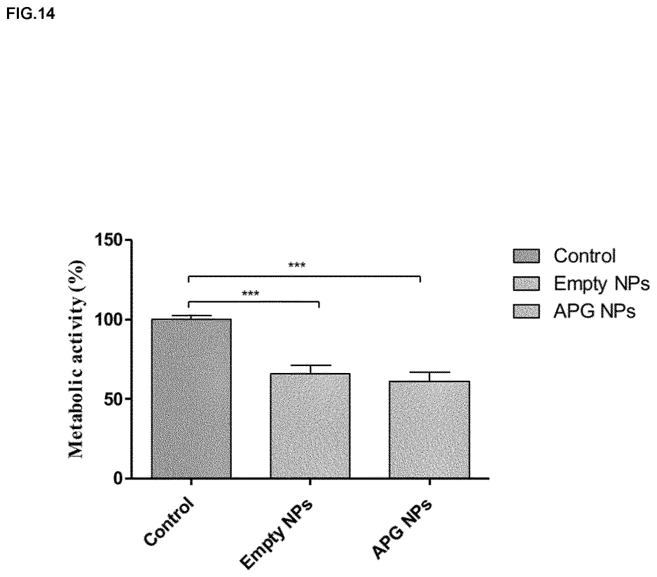

The inventors could furthermore show that both the Loaded Lipid Nanoparticles (“APG NLCs”) as well as Lipid Nanoparticles possess antibacterial metabolic reduction capacity (see Example 8 and FIG. 14). This demonstrates the antibacterial efficacy of the compositions of present invention against bacterial infections. In another aspect the present invention therefore relates to a composition comprising lipid nanoparticles as described herein for use in the treatment or prevention of bacterial infections, wherein the composition is administered topically. In a preferred embodiment of this aspect the composition is suitable for ocular topical administration. The ocular administration can be via direct administration into the eye, such as for example onto the cornea or into the conjunctival sac. In a preferred embodiment the composition is administered as an eye drop.

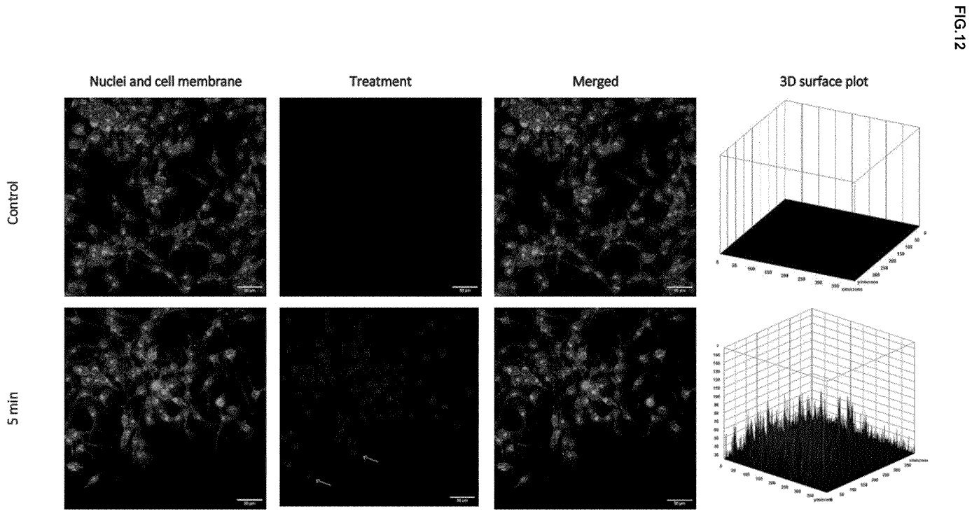

Furthermore, the inventors have studied the cytotoxic capacity of the composition of present invention in ocular tumoral cells and could show that Loaded Lipid Nanoparticles and Lipid Nanoparticles exerted a great cytotoxicity against the cancer cells in all tested concentrations, whereas free APG caused significant toxicity from 0.005-0.05 mg/mL (Example 6 and FIG. 11). It was observed that in all studied concentrations Loaded Lipid Nanoparticles showed a significantly higher antitumoral effect than free APG, most certainly due to the slow release of APG and the increased penetration of the NLCs into the cancer cells, leading to the higher cytotoxic effect. Lipid Nanoparticles also showed a significantly higher antitumoral effect than free APG.

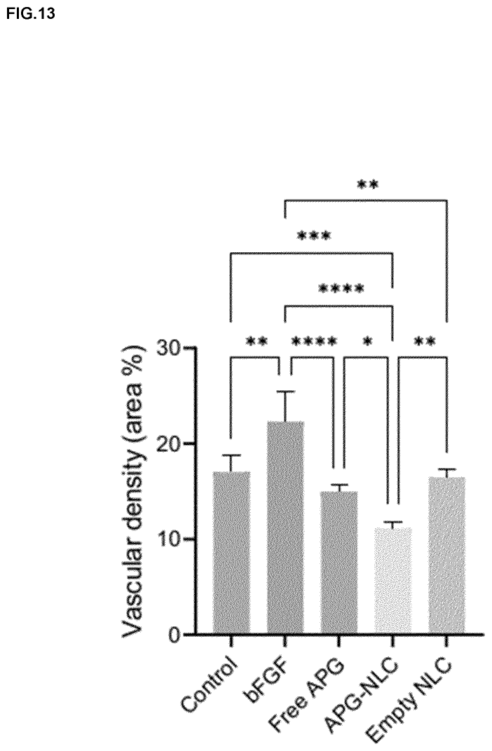

An in vitro assessment using CAM of embryonated eggs showed that Loaded Lipid Nanoparticles possess antiangiogenic efficacy, whereas free APG and Lipid Nanoparticles did not seem to have any significant effect on the blood vasculature (Example 7 and FIG. 13).

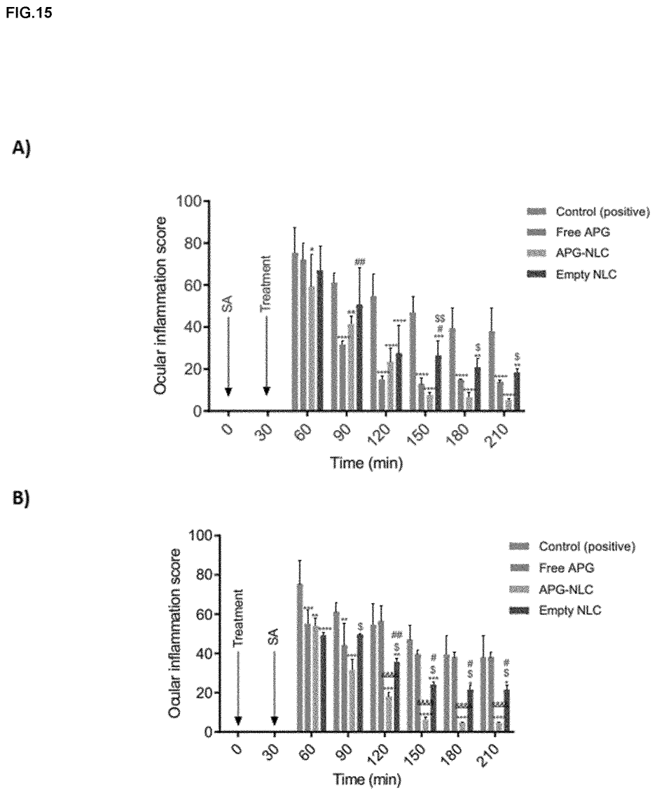

Finally, the anti-inflammatory activity of the compositions of present invention were assessed in vivo and the capacity of the NLCs to prevent and treat ocular inflammation was confirmed in two different tests (see Example 9 and FIG. 15). The tests revealed that the degree of inflammation was significantly reduced after the first 30 minutes post-administration of Loaded Lipid Nanoparticles. Lipid Nanoparticles exerted anti-inflammatory activity after 90 minutes. Loaded Lipid Nanoparticles had significantly higher anti-inflammatory effects than Lipid Nanoparticles after 2 h after application, showing the effect of the prolonged release of the APG in the NLCs.

The in vivo inflammatory prevention test showed that eyes treated with Loaded Lipid Nanoparticles presented a faster swelling reduction than eyes treated with free APG, mainly owing to tear clearance in case of free APG and the improved ocular surface adherence of lipid nanoparticles, thus presenting longer residence time in the cornea. Loaded Lipid Nanoparticles exhibited significant differences regarding positive control over the time. Lipid Nanoparticles also showed an anti-inflammatory effect in vivo. Initially, the effect was similar to the one seen with Loaded Lipid Nanoparticles, but after 90 min of treatment a significant anti-inflammatory effect produced by APG could be observed. Thus, Loaded Lipid Nanoparticles exhibited a preventive effect caused by the sustained release of APG and the synergic activity of the vehicle, which could attribute to an initial anti-inflammatory effect. Hence, it can be concluded that the controlled release system based on Loaded Lipid Nanoparticles has ocular anti-inflammatory activity, both for prevention level and inflammation treatment.

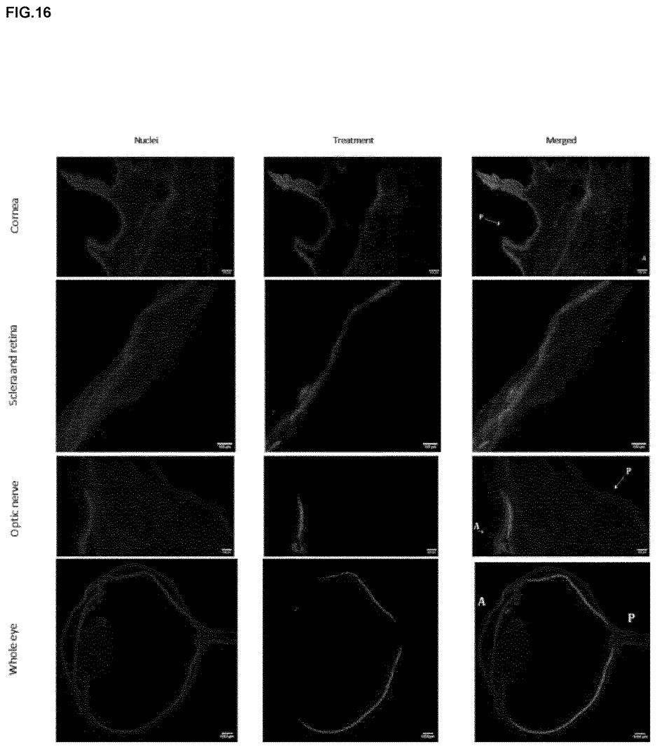

Biodistribution after topical administration of the Loaded Lipid Nanoparticles was tested showing that that after 3 h the Loaded Lipid Nanoparticles were indeed located in the eye. Specifically, Loaded Lipid Nanoparticles were able to reach the posterior segment of the eye, being distributed mainly in the retina. Moreover, Loaded Lipid Nanoparticles also had a high interaction with the cornea and the ciliary muscles (see Example 10 and FIG. 16).

### PREFERRED EMBODIMENTS OF THE LIPID NANOPARTICLES

In one preferred embodiment of the composition of present invention said lipid nanoparticles comprise:
a. 0.01-30% (w/v) rosehip oil;
b. 1-50% (w/v) solid lipid, preferably glyceryl dibehenate;
c. 0.001-20% (w/v) a cationic or non-cationic surfactant, or mixtures thereof;
d. optionally 0.00-10% (w/v) active ingredient, preferably apigenin.

In one preferred embodiment of the composition of present invention said lipid nanoparticles comprise:
a. 0.01-30% (w/v) rosehip oil;
b. 1-50% (w/v) solid lipid, preferably glyceryl dibehenate;
c. 0.001-10% (w/v) cationic surfactant; preferably DDAB;
d. 0.00-10% (w/v) optionally a non-cationic surfactant, preferably polysorbate 80;
e. optionally 0.00-10% (w/v) active ingredient, preferably apigenin.

In one preferred embodiment of the composition of present invention said lipid nanoparticles comprise:
a. 0.01-30% (w/v) rosehip oil;
b. 1-50% (w/v) solid lipid, preferably glyceryl dibehenate;
c. 0.001-10% (w/v) optionally cationic surfactant; preferably DDAB;
d. 0.00-10% (w/v) a non-cationic surfactant, preferably polysorbate 80;
e. optionally 0.00-10% (w/v) active ingredient, preferably apigenin.

In one preferred embodiment of the composition of present invention said lipid nanoparticles comprise:
a. 0.01-30% (w/v) rosehip oil;
b. 1-50% (w/v) solid lipid, preferably glyceryl dibehenate;
c. 0.001-20% (w/v) a cationic or non-cationic surfactant, or mixtures thereof.
d. 0.00-10% (w/v) active ingredient, preferably apigenin.

In one preferred embodiment of the composition of present invention said lipid nanoparticles comprise:
a. 0.01-10% (w/v) rosehip oil;
b. 1-30% (w/v) solid lipid, preferably glyceryl dibehenate;
c. 0.001-20% (w/v) a cationic or non-cationic surfactant, or mixtures thereof;
d. optionally 0.00-10% (w/v) active ingredient, preferably apigenin.

In one preferred embodiment of the composition of present invention said lipid nanoparticles comprise:
a. 0.01-10% (w/v) rosehip oil;
b. 1-30% (w/v) solid lipid, preferably glyceryl dibehenate;
c. 0.01-5.0% (w/v) cationic surfactant; preferably DDAB;
d. 0.5-7.0% (w/v) optionally a non-cationic surfactant, preferably polysorbate 80;
e. optionally 0.00-10% (w/v) active ingredient, preferably apigenin.

In one preferred embodiment of the composition of present invention said lipid nanoparticles comprise:
a. 0.01-10% (w/v) rosehip oil;
b. 1-30% (w/v) solid lipid, preferably glyceryl dibehenate;
c. 0.01-5.0% (w/v) optionally cationic surfactant; preferably DDAB;
d. 0.5-7.0% (w/v) a non-cationic surfactant, preferably polysorbate 80;
e. optionally 0.00-10% (w/v) active ingredient, preferably apigenin.

In one preferred embodiment of the composition of present invention said lipid nanoparticles comprise:
a. 0.01-10% (w/v) rosehip oil;
b. 1-30% (w/v) solid lipid, preferably glyceryl dibehenate;
c. 0.001-20% (w/v) a cationic or non-cationic surfactant, or mixtures thereof.
d. 0.00-10% (w/v) active ingredient, preferably apigenin.

In one preferred embodiment of the composition of present invention said lipid nanoparticles comprise:
a. 2.5-12.5% (w/v) rosehip oil;
b. 1-30% (w/v) solid lipid, preferably glyceryl dibehenate;
c. 0.001-10% (w/v) a cationic or non-cationic surfactant, or mixtures thereof;
d. optionally 0.00-10% (w/v) active ingredient, preferably apigenin.

In one preferred embodiment of the composition of present invention said lipid nanoparticles comprise:
a. 2.5-12.5% (w/v) rosehip oil;
b. 1-30% (w/v) solid lipid, preferably glyceryl dibehenate;
c. 0.025-0.5% (w/v) cationic surfactant; preferably DDAB;
d. 1.0-5.0% (w/v) optionally a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-12.5% (w/v) rosehip oil;
b. 1-30% (w/v) solid lipid, preferably glyceryl dibehenate;
c. 0.025-0.5% (w/v) optionally cationic surfactant; preferably DDAB;
d. 1.0-5.0% (w/v) a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-12.5% (w/v) rosehip oil;
b. 1-30% (w/v) solid lipid, preferably glyceryl dibehenate;
c. 0.001-10% (w/v) a cationic or non-cationic surfactant, or mixtures thereof.
d. 0.1-2.5% (w/v) active ingredient, preferably apigenin.

In one preferred embodiment of the composition of present invention said lipid nanoparticles comprise:
a. 2.5-12.5% (w/v) rosehip oil;
b. 1.0-6.5% (w/v) solid lipid, preferably glyceryl dibehenate;
c. 0.001-10% (w/v) a cationic or non-cationic surfactant, or mixtures thereof;
d. optionally 0.1-2.5% (w/v) active ingredient, preferably apigenin.

In one preferred embodiment of the composition of present invention said lipid nanoparticles comprise:
a. 2.5-12.5% (w/v) rosehip oil;
b. 1.0-6.5% (w/v) solid lipid, preferably glyceryl dibehenate;
c. 0.025-0.4% (w/v) cationic surfactant; preferably DDAB;
d. 3.0-5.0% (w/v) optionally a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-12.5% (w/v) rosehip oil;
b. 1.0-6.5% (w/v) solid lipid, preferably glyceryl dibehenate;
c. 0.025-0.4% (w/v) optionally cationic surfactant; preferably DDAB;
d. 3.0-5.0% (w/v) a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-12.5% (w/v) rosehip oil;
b. 1.0-6.5% (w/v) solid lipid, preferably glyceryl dibehenate;
c. 0.001-10% (w/v) a cationic or non-cationic surfactant, or mixtures thereof.
d. 0.1-2.5% (w/v) active ingredient, preferably apigenin.

In one preferred embodiment of the composition of present invention said lipid nanoparticles comprise:
a. 2.5-12.5% (w/v) rosehip oil;
b. 1.0-6.5% (w/v) solid lipid, preferably glyceryl dibehenate;
c. 0.03-0.4% (w/v) cationic surfactant; preferably DDAB;
d. 3.0-5.0% (w/v) optionally a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-12.5% (w/v) rosehip oil;
b. 1.0-6.5% (w/v) solid lipid, preferably glyceryl dibehenate;
c. 0.03-0.4% (w/v) optionally cationic surfactant; preferably DDAB;
d. 3.0-5.0% (w/v) a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-10% (w/v) rosehip oil;
b. 1.0-6.5% (w/v) solid lipid, preferably glyceryl dibehenate;
c. 0.001-10% (w/v) a cationic or non-cationic surfactant, or mixtures thereof;
d. optionally 0.1-2.5% (w/v) active ingredient, preferably apigenin.

In one preferred embodiment of the composition of present invention said lipid nanoparticles comprise:
a. 2.5-10% (w/v) rosehip oil;
b. 1.0-6.5% (w/v) solid lipid, preferably glyceryl dibehenate;
c. 0.025-0.4% (w/v) cationic surfactant; preferably DDAB;
d. 3.0-5.0% (w/v) optionally a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-10% (w/v) rosehip oil;
b. 1.0-6.5% (w/v) solid lipid, preferably glyceryl dibehenate;
c. 0.025-0.4% (w/v) optionally cationic surfactant; preferably DDAB;
d. 3.0-5.0% (w/v) a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-10% (w/v) rosehip oil;
b. 1.0-6.5% (w/v) solid lipid, preferably glyceryl dibehenate;
c. 0.001-10% (w/v) a cationic or non-cationic surfactant, or mixtures thereof.
d. 0.1-2.5% (w/v) active ingredient, preferably apigenin.

In one preferred embodiment of the composition of present invention said lipid nanoparticles comprise:
a. 2.5-5.0% (w/v) rosehip oil;
b. 1.0-6.5% (w/v) solid lipid, preferably glyceryl dibehenate;
c. 0.001-10% (w/v) a cationic or non-cationic surfactant, or mixtures thereof;
d. optionally 0.1-2.5% (w/v) active ingredient, preferably apigenin.

In one preferred embodiment of the composition of present invention said lipid nanoparticles comprise:
a. 2.5-5.0% (w/v) rosehip oil;
b. 1.0-6.5% (w/v) solid lipid, preferably glyceryl dibehenate;
c. 0.025-0.4% (w/v) cationic surfactant; preferably DDAB;
d. 3.0-5.0% (w/v) optionally a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-5.0% (w/v) rosehip oil;
b. 1.0-6.5% (w/v) solid lipid, preferably glyceryl dibehenate;
c. 0.025-0.4% (w/v) optionally cationic surfactant; preferably DDAB;
d. 3.0-5.0% (w/v) a non-cationic surfactant, preferably polysorbate 80;
e. optionally 0.1-2.5% (w/v) active ingredient, preferably apigenin.

In one preferred embodiment of the composition of present invention said lipid nanoparticles comprise:
a. 2.5-5.0% (w/v) rosehip oil;
b. 1.0-6.5% (w/v) solid lipid, preferably glyceryl dibehenate;
c. 0.001-10% (w/v) a cationic or non-cationic surfactant, or mixtures thereof.
d. 0.1-2.5% (w/v) active ingredient, preferably apigenin.

In one preferred embodiment of the composition of present invention said lipid nanoparticles comprise a. 0.01-30% (w/v) rosehip oil;
b. 1-50% (w/v) solid lipid, preferably glyceryl dibehenate;
c. 0.001-10% (w/v) cationic surfactant, preferably DDAB;
d. optionally 0.00-10% (w/v) non-cationic surfactant, preferably polysorbate 80;
e. optionally 0.00-10% (w/v) active ingredient, preferably apigenin.

In one preferred embodiment of the composition of present invention said lipid nanoparticles comprise
a. 0.01-30% (w/v) rosehip oil;
b. 1-50% (w/v) solid lipid, preferably glyceryl dibehenate;
c. 0.005-10% (w/v) cationic surfactant, preferably DDAB;
d. 0.01-10% (w/v) non-cationic surfactant, preferably polysorbate 80;
e. optionally 0.01-10% (w/v) active ingredient, preferably apigenin.

In one preferred embodiment of the composition of present invention said lipid nanoparticles comprise
a. 0.1-10% (w/v) rosehip oil;
b. 1-30% (w/v) solid lipid, preferably glyceryl dibehenate;
c. 0.01-5.0% (w/v) cationic surfactant, preferably DDAB;
d. 0.5-7.0% (w/v) non-cationic surfactant, preferably polysorbate 80;
e. optionally 0.01-10% (w/v) active ingredient, preferably apigenin.

In one preferred embodiment of the composition of present invention said lipid nanoparticles comprise
a. 2.5-12.5% (w/v) rosehip oil;
b. 1.0-30% (w/v) solid lipid, preferably glyceryl dibehenate;
c. 0.025-0.5% (w/v) cationic surfactant, preferably DDAB;
d. 1.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 2.5-12.5% (w/v) rosehip oil
b. 1.0-6.5% (w/v) solid lipid, preferably glyceryl dibehenate;
c. 0.025-0.4% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 2.5-12.5% (w/v) rosehip oil
b. 1.0-6.5% (w/v) solid lipid, preferably glyceryl dibehenate;
c. 0.025-0.4% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 2.5-12.5% (w/v) rosehip oil
b. 1.0-6.5% (w/v) solid lipid, preferably glyceryl dibehenate;
c. 0.03-0.4% (w/v) cationic surfactant, preferably DDAB;
d. 3.0-5.0% (w/v) non-cationic surfactant, preferably polysorbate 80;
e. 0.1-2.5% (w/v) apigenin.

In one preferred embodiment of the composition of present invention said lipid nanoparticles comprise
a. 2.5-12.5% (w/v) rosehip oil
b. 1.0-6.5% (w/v) solid lipid, preferably glyceryl dibehenate;
c. 0.03-0.1% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 2.5-10% (w/v) rosehip oil
b. 1.0-20% (w/v) solid lipid, preferably glyceryl dibehenate;
c. 0.025-0.4% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 2.5-10% (w/v) rosehip oil
b. 1.0-6.5% (w/v) solid lipid, preferably glyceryl dibehenate;
c. 0.025-0.4% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 2.5-5% (w/v) rosehip oil
b. 3.0-6.5% (w/v) solid lipid, preferably glyceryl dibehenate;
c. 0.025-0.1% (w/v) cationic surfactant, preferably DDAB;
d. 3.0-4.0% (w/v) non-cationic surfactant, preferably polysorbate 80;
e. optionally 0.1-2.5% (w/v) active ingredient, preferably apigenin.

In one preferred embodiment of the composition of present invention said lipid nanoparticles comprise
a. 3.0% (w/v) rosehip oil,
b. 4.5% (w/v) glyceryl dibehenate,
c. 0.05% (w/v) DDAB,
d. 3.5% (w/v) polysorbate 80,
e. optionally 0.1% (w/v) apigenin.

In one embodiment the lipid nanoparticles further comprise from 0.00001%-0.001% (w/v), preferably 0.0001% (w/v) hyaluronic acid, more preferably wherein said hyaluronic acid coats the lipid nanoparticles.

In a further aspect the present invention relates to a composition comprising lipid nanoparticles, said lipid nanoparticles comprising
a. at least one liquid lipid with anti-inflammatory properties;
b. at least one solid lipid;
c. at least one cationic surfactant
d. optionally, at least a non-cationic surfactant;
e. optionally, at least one active ingredient.

In one preferred embodiment of this aspect the composition of present invention is suitable for ocular topical administration. The ocular administration can be via direct administration into the eye, such as for example onto the cornea or into the conjunctival sac. In a preferred embodiment the composition is administered as an eye drop.

In one embodiment the at least one said liquid lipid with anti-inflammatory properties is selected from the group consisting of rosehip oil, tea tree oil, lavender oil, linoleic acid, stearic acid, palmitic acid, castor oil, safflower oil, melon seed oil, salicornia oil, evening primrose oil, poppyseed oil, grape seed oil, prickly pear oil, artichoke oil, hemp oil, wheat germ oil, cottonseed oil, corn oil, walnut oil, soybean oil, sesame oil, rice bran oil, argan oil, pistachio oil, peach oil, almond oil, canola oil, avocado oil, flaxseed oil, sunflower oil, peanut oil, palm oil, olive oil, macadamia oil, coconut oil, rosemary oil, lavender oil, Origanum vulgare oil, thyme oil, mint oil, eucalyptus oil, ginger oil, Cuminum cyminum I. oil, turmeric oil, clove oil, oleic acid, oregano oil, rose oil, fennel oil, bergamot oil, chamomile oil, helichrysum oil, patchouli oil, frankincense oil, copaiba oil, peppermint oil, black pepper oil, sweet marjoram oil, basil oil, clove oil, clary sage oil, lemongrass oil, geranium oil, wintergreen oil, cannabis oil, cannabidiol, spruce oil, niaouli oil, cardamom oil, pine tree oil, fir tree oil, juniper tree oil, verbena oil, marjoram oil, katafray oil, bitter orange oil, hypericum oil, arnica oil, coriander oil, mustard oil, perilla seed oil, Centella asiatica oil, calendula oil, laurel oil, camphor oil, cinnamon oil, oatmeal oil, docosahexaenoic acid, eicosapentaenoic acid, dandelion oil, krill oil, electrophorus electricus oil, potamotrygon motoro oil, boa constrictor oil, chelonoidis denticulate oil, Melanosuchus niger oil, inia geoffrensis oil, horse oil, anchovy oil, prunus seed oil, Tropidurus hispidus oil, emu oil, maqian fruits essential oil, fructus alpinia oil, Cinnamomum cassia essential oil, Angelica sinensis oil, Gynura procumbens oil, spirulina oil, citrus limetta oil, citrus aurantium oil, Atractylodes macrocephala oil, Artemisia argyi oil, Gynura procumbens oil, Acorus gramineus and oil, algal oil, fish oil, Zanthoxylum coreanum nakai oil, cod liver oil, Perna canaliculus oil, chia seed oil, or combinations thereof.

In one embodiment of present invention the said liquid lipid with anti-inflammatory properties is selected from derivatives of the above listed liquid lipids.

In a preferred embodiment the said liquid lipid with anti-inflammatory properties is rosehip oil.

It is to be understood that the amounts of rose hip oil specified in the above preferred embodiments of the lipid nanoparticles equally apply to any of the other lipid liquids that can be used in the nanoparticles of present invention.

In another aspect the present invention relates to a composition comprising lipid nanoparticles as described herein for use in the treatment, amelioration or prevention of bacterial infections, wherein the composition is administered topically.

In another aspect the present invention relates to a composition comprising lipid nanoparticles as described herein for use in the treatment, amelioration or prevention of ocular diseases, such as ocular inflammation, glaucoma, ocular tumors, bacterial and viral infections of the eye, age-related macular degeneration, cataracts or diabetic retinopathy, dry eye disease. In a preferred embodiment the treatment of, amelioration or prevention of DED is envisaged.

It is finally contemplated that any features described herein can optionally be combined with any of the embodiments of any product, method or medical use of the invention; and any embodiment discussed in this specification can be implemented with respect to any of these.

It will be understood that particular embodiments described herein are shown by way of illustration and not as limitations of the invention.

All publications and patent applications are herein incorporated by reference to the same extent as if each individual publication or patent application was specifically and individually indicated to be incorporated by reference.

The following examples serve to illustrate the present invention and should not be construed as limiting the scope thereof.

### EXAMPLES

### Materials

APG: Apollo Scientific (Cheshire, UK)
Compritol® 888 ATO (Glyceryl dibehenate): Gattefossé (Madrid, Spain)
Tween® 80 (Polysorbate 80), Bengal Rose and Fluorescein: Sigma Aldrich (Madrid, Spain)
Rosehip oil: Acofarma Fórmulas Magistrales (Barcelona, Spain)
Dimethyldioactadecylammonium bromide (DDAB): TCI Europe (Zwijndrecht, Belgium)
Sodium hyaluronate: Bloomage Freda Biopharm (Jinan, China)

All others chemical reagents and components used in this research were of analytical grade.

A Millipore Milli-Q Plus system was used to obtain purified water.

### Example 1: Loaded Lipid Nanoparticles Preparation and Optimization

### 1.1 Preparation, Optimization and Physicochemical Characterization

The production of Loaded Lipid Nanoparticles was carried out by high-pressure homogenization method (Homogenizer FPG 12800, Stansted, United Kingdom) after generating a primary emulsion with the mixture of components with an Ultraturrax T25 (IKA, Germany) at 8000 rpm for 30 s. The production conditions were 85° C., three homogenization cycles and 900 bars of pressure. A design of experiments approach (DoE) used in order to optimize formulation parameters. A central composite factorial design (containing 2 replicated centre points, 16 factorial points and 8 axial points) was developed using statistical program Statgraphics Centurion 18® version 18.1.12 software (Virginia, USA). Four independent variables: APG concentration (%), surfactant concentration (%), glyceryl dibehenate+rosehip oil concentration (%) and glyceryl dibehenate concentration contained in the mixture glyceryl dibehenate+rosehip oil (%) were evaluated to determine their influence on the NLCs properties. Mean particle diameter size (Zₐᵥ), polydispersity index (PDI), entrapment efficiency (EE) and Zeta potential (ZP) were designated as the dependent variables. Once the optimized formulation was obtained, increasing amounts of a cationic surfactant were added in order to obtain a cationic surface charge. Then, sodium hyaluronate was added to the formulation [15]. Zₐᵥ and PDI were assessed by photon correlation spectroscopy (PCS) with a ZetaSizer Nano ZS (Malvern Instruments, Malvern, UK). ZP was estimated by electrophoretic mobility. For these measurements, samples were diluted with Milli-Q water 1:10 and analysed by triplicate at 25° C. EE was determined indirectly. Previously to the analysis, the non-loaded drug was separated from NPs by filtration/centrifugation at 14,000 r.p.m. (Mikro 22 Hettich Zentrifugen, Germany) using an Amicon® Ultra 0.5 centrifugal filter device (Amicon Millipore Corporation, Ireland). The encapsulation efficiency (EE) was calculated by the difference between the total amount of drug and the free drug, present in the filtered fraction, using Eq. 1 [16]:

$$EE ( \% ) = \frac{Total amount of APG - Free APG}{Total amount of APG} \qquad Eq . 1$$

The quantification of APG was performed by a modified reverse-phase high-performance liquid chromatography (RP-HPLC). Briefly, samples were quantified using HPLC Waters 2695 (Waters, Massachusetts, USA) separation module and a Kromasil® C18 column (5 μm, 150 ×4.6 mm) with a mobile phase formed by a water phase of 2% acetic acid and an organic phase of methanol, in a gradient (from 40% to 60% of water phase in 5 min and back in next 5 min) at a flow rate of 0.9 ml/min. A diode array detector Waters® 2996 at a wavelength of 300 nm was used to detect the APG and data were processed using Empower 3® Software.

Table 1 shows the effect of independent variables used on dependent variables analysed and the values obtained. Zₐᵥ values are mostly around 200 nm. The developed Loaded Lipid Nanoparticles exhibit a negative surface charge ZP<−20 mV. In all cases EE was higher than 95%, thus meaning that APG was completely encapsulated. Most of the formulations have PDI values below 0.3, indicating a homogeneous distribution of nanoparticles.

TABLE 1
Design of experiments and characterization of the different formulations developed.

| Independent variables | | | | | | | |
|---|---|---|---|---|---|---|---|
| | Liquid | Solid | Polysorbate | Dependent variables | | | |
| APG (%) | Lipid (%) | Lipid (%) | 80 (%) | Zₐᵥ ± SD (nm) | PDI ± SD | ZP ± SD (mV) | EE ± SD (%) |

Factorial points

| | | | | | | | | | | | | |
|---|---|---|---|---|---|---|---|---|---|---|---|---|
| A1 | −1 | 1 | −1 | 0.75 | 1 | 4.25 | −1 | 2 | 176.1 ± 2.3 | 0.278 ± 0.009 | −21.7 ± 0.7 | 99.9 ± 0.1 |
| A2 | 1 | 2 | −1 | 1.75 | −1 | 3.25 | 1 | 4 | 221.5 ± 2.5 | 0.313 ± 0.012 | −21.4 ± 0.5 | 96.7 ± 0.1 |
| A3 | 1 | 2 | 1 | 3.50 | −1 | 6.50 | −1 | 2 | 253.9 ± 4.4 | 0.246 ± 0.012 | −22.6 ± 0.3 | 99.9 ± 0.1 |
| A4 | −1 | 1 | 1 | 1.50 | 1 | 8.50 | −1 | 2 | 271.4 ± 5.5 | 0.273 ± 0.008 | −23.0 ± 0.8 | 99.9 ± 0.1 |
| A5 | −1 | 1 | 1 | 3.50 | −1 | 6.50 | −1 | 2 | 243.7 ± 0.7 | 0.236 ± 0.014 | −24.6 ± 0.5 | 99.7 ± 0.3 |
| A6 | −1 | 1 | 1 | 3.50 | −1 | 6.50 | 1 | 4 | 214.7 ± 1.6 | 0.181 ± 0.010 | −18.2 ± 0.9 | 99.9 ± 0.1 |
| A7 | −1 | 1 | −1 | 0.75 | 1 | 4.25 | 1 | 4 | 165.9 ± 1.2 | 0.265 ± 0.012 | −17.6 ± 0.2 | 99.9 ± 0.1 |
| A8 | −1 | 1 | −1 | 1.75 | −1 | 3.25 | −1 | 2 | 161.2 ± 2.3 | 0.255 ± 0.023 | −22.3 ± 0.4 | 95.1 ± 0.2 |
| A9 | 1 | 2 | 1 | 4.50 | −1 | 6.50 | 1 | 4 | 212.2 ± 1.8 | 0.217 ± 0.019 | −19.6 ± 0.2 | 99.9 ± 0.1 |
| A10 | −1 | 1 | 1 | 1.50 | 1 | 8.50 | 1 | 4 | 236.8 ± 2.0 | 0.244 ± 0.017 | −18.4 ± 0.5 | 99.9 ± 0.1 |
| A11 | 1 | 2 | −1 | 0.75 | 1 | 4.25 | 1 | 4 | 191.9 ± 2.7 | 0.318 ± 0.058 | −19.2 ± 0.6 | 99.9 ± 0.1 |
| A12 | −1 | 1 | −1 | 1.75 | −1 | 3.25 | 1 | 4 | 202.7 ± 2.4 | 0.203 ± 0.002 | −18.7 ± 0.2 | 97.7 ± 0.1 |
| A13 | 1 | 2 | 1 | 1.50 | 1 | 8.50 | −1 | 2 | 304.6 ± 5.7 | 0.276 ± 0.007 | −20.1 ± 0.4 | 99.1 ± 0.1 |
| A14 | 1 | 2 | −1 | 1.75 | −1 | 3.25 | −1 | 2 | 188.7 ± 2.3 | 0.374 ± 0.008 | −22.2 ± 0.4 | 99.9 ± 0.1 |
| A15 | 1 | 2 | 1 | 1.50 | 1 | 8.50 | 1 | 4 | 240.5 ± 2.5 | 0.277 ± 0.015 | −18.0 ± 0.4 | 99.6 ± 0.1 |
| A16 | 1 | 2 | −1 | 0.75 | 1 | 4.25 | −1 | 2 | 200.5 ± 2.8 | 0.425 ± 0.015 | −22.4 ± 0.6 | 99.9 ± 0.1 |

Axial points

| | | | | | | | | | | | | |
|---|---|---|---|---|---|---|---|---|---|---|---|---|
| A17 | −2 | 0.5 | 0 | 1.857 | 0 | 5.625 | 0 | 3 | 181.2 ± 1.4 | 0.221 ± 0.029 | −19.5 ± 0.3 | 99.9 ± 0.1 |
| A18 | 2 | 2.5 | 0 | 1.875 | 0 | 5.625 | 0 | 3 | 230.3 ± 2.7 | 0.327 ± 0.007 | −20.2 ± 0.2 | 99.9 ± 0.1 |
| A19 | 0 | 1.5 | −2 | 0.625 | 0 | 1.875 | 0 | 3 | 202.0 ± 3.9 | 0.556 ± 0.015 | −22.3 ± 0.3 | 99.9 ± 0.1 |
| A20 | 0 | 1.5 | 2 | 3.125 | 0 | 9.375 | 0 | 3 | 280.9 ± 5.7 | 0.259 ± 0.009 | −20.7 ± 0.4 | 99.9 ± 0.1 |
| A21 | 0 | 1.5 | 0 | 4.50 | −2 | 5.50 | 0 | 3 | 187.3 ± 1.3 | 0.195 ± 0.012 | −20.4 ± 0.5 | 99.9 ± 0.1 |
| A22 | 0 | 1.5 | 0 | 0.50 | 2 | 9.50 | 0 | 3 | 202.5 ± 3.3 | 0.331 ± 0.026 | −20.7 ± 0.2 | 99.9 ± 0.1 |
| A23 | 0 | 1.5 | 0 | 2.50 | 0 | 7.50 | −2 | 1 | 271.0 ± 4.3 | 0.231 ± 0.020 | −24.1 ± 0.7 | 99.9 ± 0.1 |
| A24 | 0 | 1.5 | 0 | 2.50 | 0 | 7.50 | 2 | 5 | 239.1 ± 1.6 | 0.182 ± 0.027 | −17.2 ± 0.8 | 98.1 ± 0.3 |

Central points

| | | | | | | | | | | | | |
|---|---|---|---|---|---|---|---|---|---|---|---|---|
| A25 | 0 | 1.5 | 0 | 1.875 | 0 | 5.625 | 0 | 3 | 202.0 ± 0.8 | 0.267 ± 0.012 | −20.3 ± 0.4 | 99.9 ± 0.1 |
| A26 | 0 | 1.5 | 0 | 1.875 | 0 | 5.625 | 0 | 3 | 199.3 ± 0.7 | 0.292 ± 0.038 | −19.6 ± 0.1 | 99.9 ± 0.1 |

As it can be appreciated in FIG. 3, the four variables studied had a significant effect on the formulation of the NLCs. Average size and PDI of Loaded Lipid Nanoparticles are directly influenced by the concentration of glyceryl dibehenate+rosehip oil. Higher concentrations of glyceryl dibehenate+rosehip oil provided bigger Loaded Lipid Nanoparticles but lower PDI (FIGS. 3A, 3B). ZP is influenced by the amount of surfactant, a higher concentration of surfactant, less superficial charge is obtained (FIG. 3C). EE is highly influenced by the proportion of glyceryl dibehenate added to the formulation. At higher glyceryl dibehenate concentrations, lower encapsulation of APG is obtained (FIG. 3D). With these trends, the optimized formulation contains 0.1% of APG, 4.5% glyceryl dibehenate, 3.0% rosehip oil, and 3.5% of non-cationic surfactant.

To the optimized formulation, increasing amounts of the cationic surfactant were added (Table 2). The optimized formulation was chosen based on the physicochemical parameters, where values of ZP higher than 20 mV and PDI lower than 0.3 were selected. Because of that fact, the cationic optimized formulation was 0.05% of cationic surfactant.

TABLE 2
Effect of cationic surfactant on the physicochemical parameters.

| DDAB (%) | Zₐᵥ ± SD (nm) | PDI ± SD | ZP ± SD (mV) |
|---|---|---|---|
| 0.025 | 301.8 ± 2.0 | 0.186 ± 0.003 | −0.8 ± 0.1 |
| 0.05 | 146.8 ± 0.8 | 0.253 ± 0.003 | 21.2 ± 0.4 |
| 0.06 | 144.3 ± 2.4 | 0.326 ± 0.036 | 27.3 ± 1.0 |
| 0.075 | 150.0 ± 3.8 | 0.363 ± 0.040 | 29.3 ± 0.5 |
| 0.1 | 148.2 ± 1.5 | 0.427 ± 0.018 | 35.6 ± 0.8 |
| 0.3 | 156.6 ± 1.9 | 0.407 ± 0.011 | 54.8 ± 1.6 |
| 0.5 | 147.7 ± 0.3 | 0.382 ± 0.009 | 54.4 ± 0.6 |

To this, 0.0001% of HA was added and then, the optimized Loaded Lipid Nanoparticles were obtained (Table 3).

TABLE 3
Composition and physicochemical parameters of optimized
formulation, Loaded Lipid Nanoparticles
Composition of Loaded Lipid Nanoparticles

| | | |
|---|---|---|
| APG | 0.1% | (w/v) |
| Glyceryl dibehenate | 4.5% | (w/v) |
| Rosehip oil | 3.0% | (w/v) |
| Polysorbate 80 | 3.5% | (w/v) |
| DDAB | 0.05% | (w/v) |
| HA | 0.0001% | (w/v) |

Physycochemical parameters of Loaded Lipid Nanoparticles

| Zₐᵥ ± SD (nm) | PI ± SD | ZP ± SD (mV) | EE ± SD (%) |
|---|---|---|---|
| 143.6 ± 1.3 | 0.254 ± 0.009 | 21.8 ± 0.6 | 99.9 ± 0.1 |

### 1.2 Characterization of Optimized Loaded Lipid Nanoparticles

### 1.2.1 Transmission Electron Microscopy

Transmission electron microscopy (TEM) was used to investigate the morphology of the Loaded Lipid Nanoparticles on a Jeol 1010 (Jeol USA, Dearborn Road, Peabody, MA 01960, USA). Copper grids were activated with UV light and samples were diluted (1:10) and placed on the grid surface to visualize the particles. Samples were previously subjected to negative staining with uranyl acetate (2%) [17].

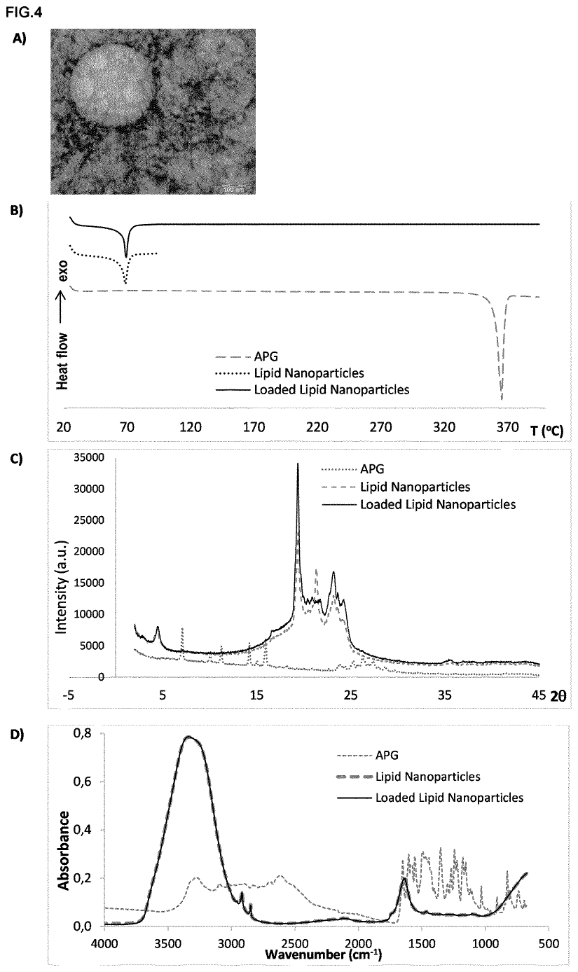

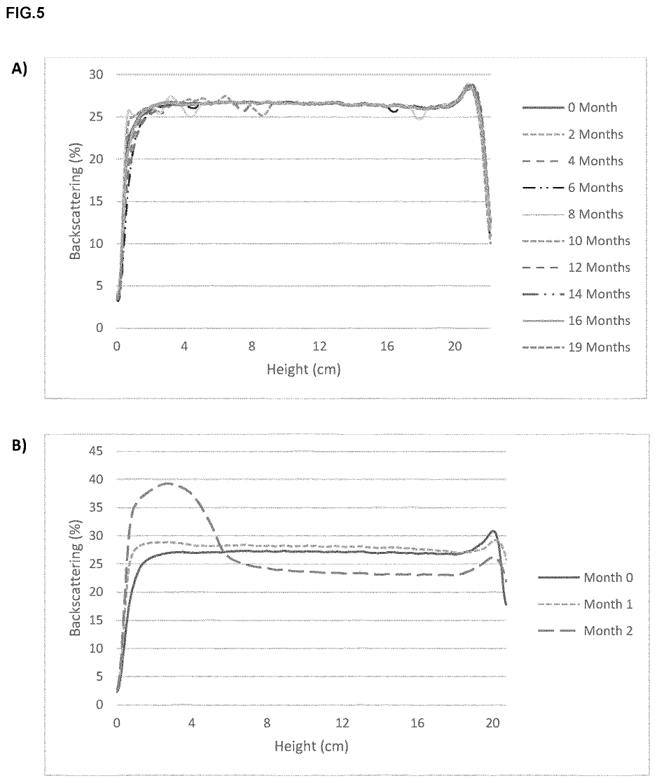

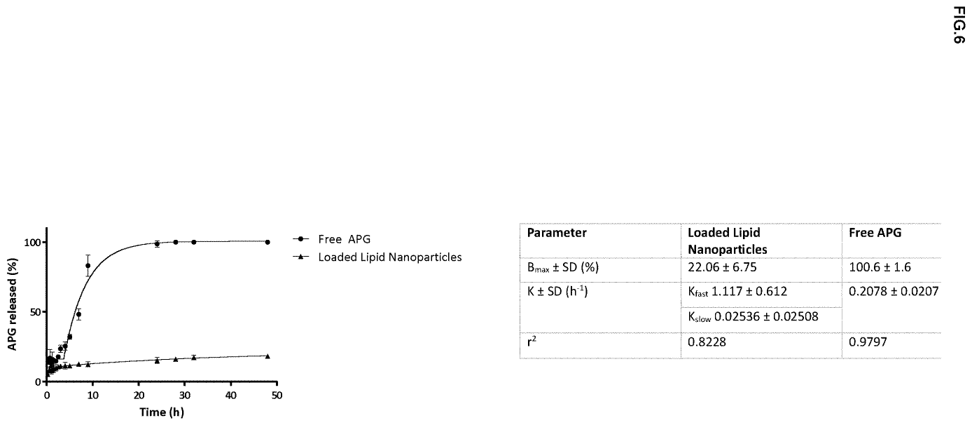

The morphology of Loaded Lipid Nanoparticles obtained by TEM shows almost spherical and soft shapes and the size below 200 nm is consistent with the results found by PCS. Particle aggregation phenomena is not observed (FIG. 4A).

### 1.3.2 Interaction Studies

Interaction studies were carried out with the formulation without HA.

Differential scanning calorimetry (DSC) analysis was performed using a DSC 823e System Mettler-Toledo, Barcelona, Spain. A pan with indium (purity ≥99.95%; Fluka, Switzerland) was used to check the calibration of the calorimetric system. An empty pan served as a reference. The DSC measurements were carried out in the Loaded Lipid Nanoparticles formulations using a heating ramp from 25 to 105° C. at 10° C./min in a nitrogen atmosphere. Data was evaluated using the Mettler STARe V 9.01 dB software (Mettler-Toledo, Barcelona, Spain).

X-ray spectroscopy (XRD) was used to analyse the amorphous or crystalline state of the samples.

Samples were sandwiched between 3.6 μm polyester films and exposed to CuKα radiation (45 kV, 40 mA, λ=1.5418 Å) in the range (2θ) of 2-60° with a step size of 0.026° and a measuring time of 200 s per step.

Fourier transform infrared (FTIR) spectra of Loaded Lipid Nanoparticles were obtained using a Thermo Scientific Nicolet iZ10 with an ATR diamond and DTGS detector (Barcelona, Spain) [18].

DSC was carried out in order to study the crystallinity and the melting point variations of the lipid mixtures and Loaded Lipid Nanoparticles. Thermogram (FIG. 4B) shows endothermal peaks, of 70.37° C. for lipid mixture, 69.78° C. for lipid mixture-APG and 69.16° C. Loaded Lipid Nanoparticles. Melting point of Loaded Lipid Nanoparticles is slightly lower because of its small size and the addition of a surfactant (Polysorbate 80) in the formulation. The peaks move to slightly lower temperatures when APG is added and the enthalpy is similar between the lipid mixture and lipid mixture-APG being ΔH Lipid mixture=82.69 Jg⁻¹, ΔH Lipid mixture-APG=84.03 Jg⁻¹ and a smaller enthalpy for the nanoparticles, being ΔH Loaded Lipid Nanoparticles=54.11Jg⁻¹. APG melting transition is characterized by an endothermal peak at 365.5° C. (ΔH=198.5 Jg⁻¹) followed by decomposition.

XRD profiles in FIG. 4C show the physical state of APG incorporated in NLCs. Intense and sharp peaks for APG and for the solid mixture of lipids are shown, indicating that these components have a crystalline structure. The peaks found for APG are not detected in Loaded Lipid Nanoparticles profile, which could mean that the drug is present in a dissolved state in the NLCs (molecular dispersion). The crystallinity of the structure of all the components was studied. The lipid mixture shows three peaks in 19.34 (2θ), i.e. d=0.46 nm indicating the most stable form of triacylglycerols, the 0 form, 21,28 (2θ) i.e. d=0.42 nm and 23,43 (2θ) i.e. d=0.38 nm, indicating the second stable form of triacylglycerols, the 0′ form. The Loaded Lipid Nanoparticles profile shows also the three peaks, which could indicate a good stability of the formulation.

FTIR analysis was used to study the interactions between the drug, the surfactant and lipid mixture (FIG. 4D). The FTIR spectra of pure APG presented vibrational bands with characteristic peak at the wave number of 3278 cm⁻¹ for O—H group. However, C—H group presented multiple small peaks at 2800 cm⁻¹. The characteristic peaks at 1650 and 1605 cm⁻¹ were obtained for the C-O functional group [19]. There was no evidence of strong bonds between APG and lipid mixture and the surfactant. APG peaks were not found in the NLCs. These results meaning that APG was encapsulated in the NLCs.

### Example 2: Stability Studies

Loaded Lipid Nanoparticles were stored at 4 and 25° C. during several months. The study was assessed analysing light backscattering (BS) profiles by a Turbiscan® Lab equipment. A glass measurement cell containing 20 mL of sample was used. Data were acquired every 30 days. The radiation source used was pulsed near-infrared light-emitting diode LED (λ=880 nm), the signal was detected by a BS detector at an angle of 45° from the incident beam. At the same time interval, values of Zₐᵥ, PDI, ZP and EE were measured.

Stability studies were carried out by the backscattering (BS) profiles of each sample at different temperatures. BS profiles provides information of destabilization mechanisms in the media, such as sedimentation, agglomeration, or aggregation [20]. In this way, BS profiles of Loaded Lipid Nanoparticles were studied at 4° C. and 25° C. The Loaded Lipid Nanoparticles formulation is stable at 4° C. for a period of 25 months, while at 25° C. the stability endures 2 months. The physicochemical parameters kept constant at 4° C. for all the study. The best storage temperature is at 4° C.

### Example 3: Biopharmaceutical Behaviour

The in vitro APG release test for Loaded Lipid Nanoparticles was performed using Franz-type diffusion cells (Crown Glass, NY, USA) with a diffusion area of 0.20 cm² and dialysis membranes of cellulose (MWCO 12 kDa). A solution of PBS with 5% polysorbate 80 and 20% ethanol under continuous stirring was used as a receptor medium assuring sink conditions (ability of the medium to dissolve the expected amount of drug) [21]. The formulations were compared with free-APG solution. The assay was carried out at 32±0.5° C. along 48 h. 300 μL of each formulation were added to the donor compartment by direct contact with the membrane. At a certain timepoints, 150 μL of sample were collected with a syringe and the volume withdrawn was replaced with receptor solution. Drug content of the samples was analysed with HPLC. The test was performed by triplicate, and the cumulative amount of APG was calculated.

The in vitro release profile of APG from the NLCs demonstrates that the formulation has a kinetic profile that is characteristic of prolonged drug release formulations. The release best fit for NLCs was two phase decay. FIG. 6 shows a faster release of APG from the NLCs during the first 8 h, after that the speed was decreased. The free APG had a faster release, achieving a 100% at 24 h while the Loaded Lipid Nanoparticles released less than 20%. It has been reported that modifying the surface of nanocarriers with hyaluronic acid can restrict water diffusion into the carrier matrix which subsequently slows down the drug release process [22]. These results indicated the formulation had a prolonged release of APG.

### Example 4: Ocular Tolerance

### 4.1 In Vitro Study: HET-CAM Test and TBS

In vitro ocular tolerance was assessed using the HET-CAM test to ensure that the formulations of Loaded Lipid Nanoparticles were non-irritating when administered as eye-drops. Irritation, coagulation, and haemorrhage phenomena were measured by applying 300 μL of the formulation studied on chorioallantoic membrane of a fertilized chicken egg and monitoring it during the first 5 min after the application. This assay was conducted according to the guidelines of ICCVAM (The Interagency Coordinating Committee on the Validation of Alternative Methods). The development of the test was carried out using 3 eggs for each group (free APG, Loaded Lipid Nanoparticles, positive control (NaOH 0.1 M) and negative control (0.9% NaCl)). The ocular irritation index (OII) was calculated by the sum of the scores of each injury according to the following expression (Eq 3) [16]:

$$OII = \frac{( 301 - H ) \cdot 5}{300} + \frac{( 301 - V ) \cdot 7}{300} + \frac{( 301 - C ) \cdot 9}{300} \qquad Eq . 3$$

where H, V and C are times (s) until the start of haemorrhage (H), vasoconstriction (V) and coagulation (C), respectively. The formulations were classified according to the following: OII≤0.9 non-irritating; 0.9<OII≤4.9 weakly irritating; 4.9<OII≤8.9 moderately irritating; 8.9<OII≤21 irritating.

Furthermore, at the end of the HET-CAM experiment, in order to quantify the damage of the membrane, trypan blue staining (TBS) was applied. The CAM was treated with 1000 μL of 0.1% trypan blue solution for 1 min. Excess dye was rinsed off with distilled water. The dyed CAM was excised and extracted with 5 mL formamide, and the absorbance of the extract was measured spectrophotometrically at 595 nm. The absorbed trypan blue was determined from a calibration curve of trypan blue in formamide [23].

HET-CAM test was applied showing that the positive controls (NaOH 1 M) resulted in severe haemorrhage, which increased over five minutes grading this solution as a severe irritant. On the other hand, application of the formulations to the chorioallantoic membrane did not cause irritation and therefore, the formulations were classified as non-irritant. Moreover, TBS quantitative results supported the results of the HET-CAM test, where Loaded Lipid Nanoparticles were non-irritant while free APG resulted irritant.

### 4.2 In Vivo Study: Draize Test

All of the procedures were approved by the Ethical Committee for Animal Experimentation of the UB and current legislation (Decree 214/97, Gencat). The formulations were evaluated using primary eye irritation test of Draize to ensure the results obtained from the HEM-CAM test. For this experiment, New Zealand male albino rabbits (2.0-2.5 kg, San Bernardo farm, Navarra, Spain) were used. 50 μL of each sample were instilled in the ocular conjunctival sac (n=3/group) and a mild massage was applied to guarantee the passage of the sample through the eyeball. The possible appearance of irritation signs (corneal opacity and area of corneal involvement, conjunctival hyperemia, chemosis, ocular discharges, and iris abnormalities) was observed at the time of instillation and after 1 h from its application and if necessary, at predefined intervals: 24 h, 48 h, 72 h, 7 days, and 21 days after administration. The opposite untreated eye was used as a negative control. Draize test score was determined directly by observing the anterior segment of the eye and changes in the structures of the cornea (turbidity or opacity), iris and conjunctiva (congestion, chemosis, swelling and secretion) [2].

The tests were carried out with free APG, Lipid Nanoparticles, Loaded Lipid Nanoparticles. In this sense, none of the developed NLCs were irritant in vivo or in vitro, while the free APG resulted irritant in vitro and non-irritant in vivo but caused an initial discomfort. These results confirmed the non-irritant potential of APG loaded lipid nanoparticles, meanwhile the free APG can induce some discomfort.

### Example 5: In Vivo Dry Eye Disease Efficacy Studies

In order to evaluate the potential to treat dry eye of Loaded Lipid Nanoparticles, Schirmer test, fluorescein and Bengal rose assessments were performed against Lipid Nanoparticles, free APG and 0.15% hyaluronic acid (commercial solution Hyabak®).

### 5.1 Induction and Treatment of Dry Eye

Twelve male New Zealand white rabbits (purchased from Livestock Research Institute, Council of Agriculture, Executive Yuan, Taiwan) weighing between 2.0 and 2.5 kg were used for the study. The rabbits were randomly divided into 4 groups: Lipid Nanoparticles, Loaded Lipid Nanoparticles, free APG solution and 0.15% hyaluronic acid (commercial solution (Hyabak®). All rabbits were housed at a room temperature of 23±2° C. with relative humidity 75±10% and alternating 12-hour light-dark cycles (8 a.m. to 8 p.m.). Both eyes of each rabbit were treated twice-daily by a topical administration of 0.1% benzalkonium chloride (BAC) drops for 2 weeks. On day 14, DES was confirmed by Schirmer test, fluorescein and Bengal rose staining. The treatment began after the confirmation of DES, where on eye of each rabbit was chosen randomly for twice-daily topical administration of Lipid Nanoparticles, Loaded Lipid Nanoparticles, free APG solution and a commercial solution. After one week of treatment, Schirmer test, fluorescein and Bengal rose staining were performed [2].

### 5.1.1. Measurement of Aqueous Tear Production

Tear production was measured using Schirmer test strips. After the topical application of anaesthetic drops (1 mg/mL tetracaine hydrochloride/4 mg/mL oxybuprocaine hydrochloride), the lower eyelid was pulled down, and a Schirmer paper strip was placed on the palpebral conjunctiva near the junction of the middle and outer thirds of the lower eyelid. After 5 min, the wetted length (mm) of the paper strips was recorded [2].

A severe decrease in the aqueous tear secretion was achieved after the application of benzalkonium chloride for 2 weeks. FIG. 7 shows the differences between each group of treatment. Lipid Nanoparticles and Loaded Lipid Nanoparticles were able to increase the tear flow in the animals, with statistically significant differences against dry eye group, p<0.01 and p<0.0001 respectively. Otherwise, free APG and the commercial solution did not improve the tear flow. Loaded Lipid Nanoparticles showed statically significant differences between all the groups: p<0.01 against Lipid Nanoparticles, and p<0.0001 against free APG and commercial solution. Lipid Nanoparticles showed statistically significant differences between free APG group (p<0.01). Loaded Lipid Nanoparticles were the treatment that attained the best score in the Schirmer test, followed by the Lipid Nanoparticles, meaning that both were able to restore the tear secretion of the animals. These results reveal the potential of the composition of the nano formulations because of the restoring of the tear flow in the animals. Loaded Lipid Nanoparticles presented a better score due to the encapsulation of APG, which it has anti-inflammatory properties, leading to a better improvement of one of the symptoms of DED.

### 5.1.2. Fluorescein Staining on the Ocular Surface

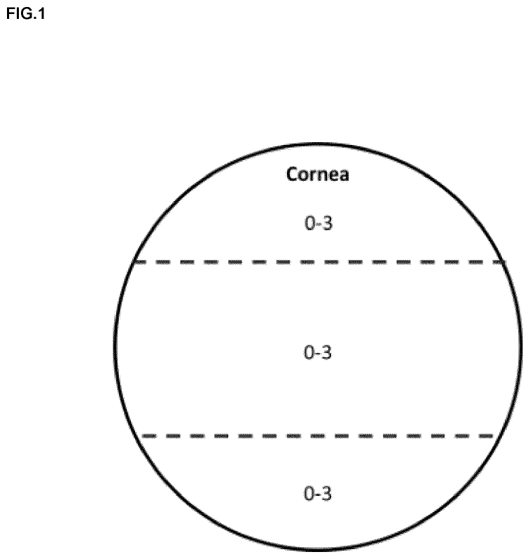

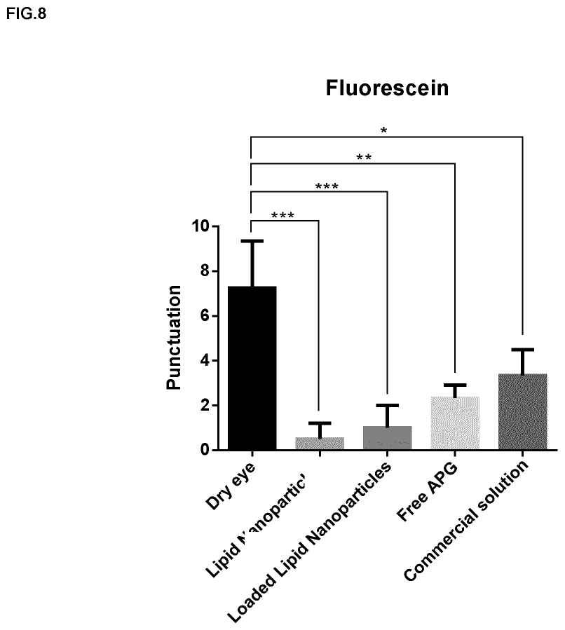

Fluorescein staining is an effective method for ocular surface evaluation. Fluorescein staining is the result of uptake caused by the disruption of corneal epithelial cell-cell junctions or damaged corneal epithelial cells [24]. Corneal fluorescein staining was performed after 2 μL of 1% fluorescein sodium were dropped into the conjunctival sac for 2 min. The ocular surface was examined under a slit lamp microscope with a cobalt blue filter. The images were collected by a digital camera (FIG. 1) and punctuated according to the stained score (9: maximum score; 0: minimum) [25]. FIG. 8 shows the differences between each group of treatment. There were statistically significant differences between all the groups against dry eye control. However, the two treatments that had a better improvement of the ocular corneal surface were Lipid Nanoparticles and Loaded Lipid Nanoparticles (p<0.001). Moreover, free APG solution and the commercial solution presented worse punctuation in the staining, meaning that both did not have the ability to restore the corneal surface as Lipid Nanoparticles and Loaded Lipid Nanoparticles.

### 5.1.3. Bengal Rose Staining on the Ocular Surface

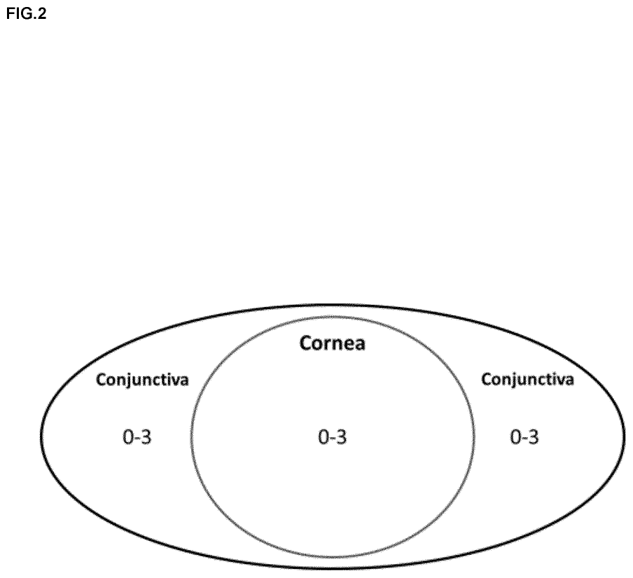

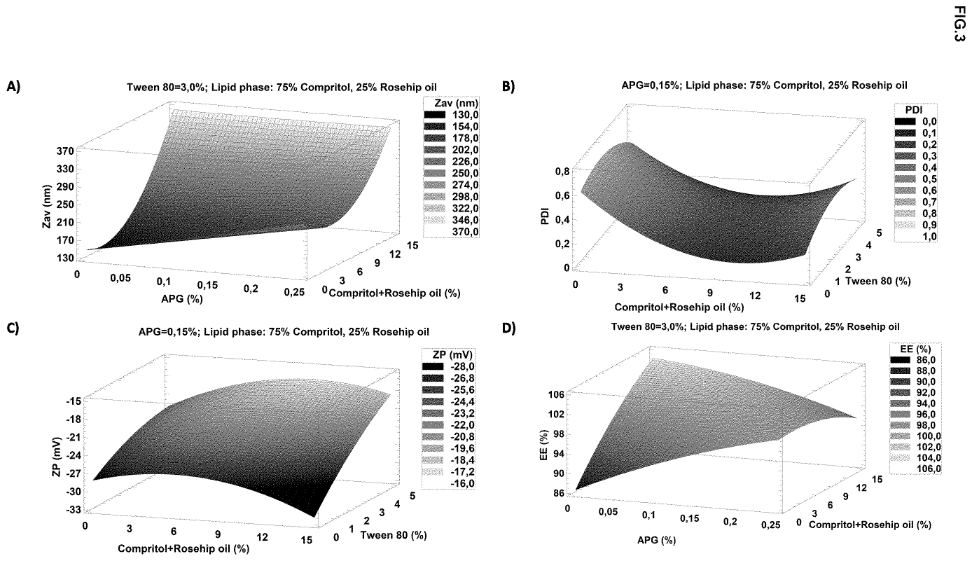

Bengal rose is an effective method to evaluate the tear film integrity. Bengal rose has been demonstrated to stain corneal and conjunctival epithelial cells that are not adequately protected by the preocular tear film. It can stain live and dead cells if they are not protected by an intact mucin layer [24]. Ocular fluorescein staining was performed after 2 μL of 0.1% Bengal rose were dropped into the conjunctival sac for 2 min. The ocular surface was examined under a slit lamp microscope with a white light. The images were collected by a digital camera. Using the Van Bijsterveld grading system, the scores were graded after 15 seconds (FIG. 2) (9: maximum score; 0: minimum) [24].

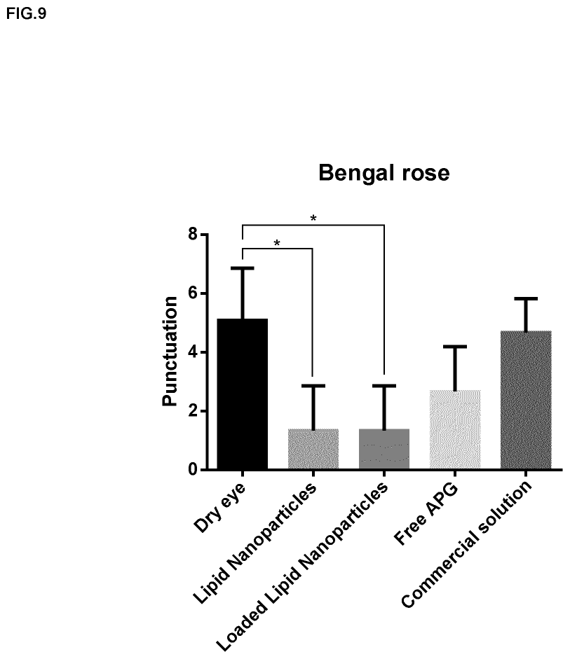

There were statistically significant differences between dry eye and Lipid Nanoparticles and Loaded Lipid Nanoparticles intensity (FIG. 9). The results showed that only NLCs were able to restore tear film in the animals. The worst punctuation for the Bengal rose staining was the commercial solution. Thus, indicating that Loaded Lipid Nanoparticles were able to restore the tear film.

These results showed the ability of the lipid nanoparticles to revert the symptoms of the DED. Lipid Nanoparticles and Loaded Lipid Nanoparticles were able to improve all the studied parameters of DED. These results could be due to the novel composition of the lipid nanoparticles. The addition of the rosehip oil provides anti-inflammatory and antioxidant properties to the Lipid Nanoparticles [26,27]. Because of this fact, Lipid Nanoparticles improves the score of both different staining of the ocular surface, showing interesting properties against DED. The addition of the APG had improved the tear secretion on the Schirmer test, which could mean that NLCs protects APG to be released and enhance the anti-inflammatory properties of the formulation, improving the DED symptomatology.

### Example 6: Cellular Experiments

### 6.1. Cell Cultures

Human corneal epithelial cells (HCE-2) (LGC Standards, Barcelona, Spain) were cultured in keratinocyte serum-free growth medium (SFM; Life Technologies, Invitrogen, GIBCO®, Paisley, UK)). It was supplemented with bovine pituitary extract 0.05 mg·mL⁻¹ and epidermal growth factor 5 ng·mL⁻¹ containing insulin 0.005 mg·mL⁻¹ and penicillin 100 U·mL⁻¹ plus streptomycin 100 mg·mL⁻¹. Cells were grown on a culture flask to 80% confluency in a humidified 10% CO₂ atmosphere at 37° C. Human UM 92-1 cells were maintained in RPMI-1640 medium, added with 10% fetal bovine serum (FBS), 2 mM L-glutamine, 100 units/mL penicillin and 100 pg-mL⁻¹ streptomycin. For MTT assay, passages from 10 to 13 were used. All the cells were incubated at 37° C. and 10% CO₂ [28].

### 6.2. Cell Viability

Cytotoxicity of Loaded Lipid Nanoparticles and Lipid Nanoparticles was determined by MTT (3-(4,5-Dimethylthiazol-2-yl)-2,5-diphenyl tetrazolium bromide) assay, by reduction of tetrazolium salt by intracellular dehydrogenases of viable living cells. For this, 100 μL of a cell suspension of 2×10⁵ cells-mL⁻¹ (for HCE-2 cells) or 1×10⁴ cells·mL⁻¹ (for UM 92-1 cells) was seeded in a 96-well plate and incubated for 48 h at 37° C. in the appropriate complete medium before treatment. Cells were incubated with samples at different concentrations (1×10³-0.1 mg·mL⁻¹) for 5, or 15 min to simulate the real conditions of the cornea (for HCE-2 cells) or 24 h (for UM 92-1 cells). Then, the medium was removed and MTT (Sigma-Aldrich Chemical Co, St. Louis, MO, USA) was added at 0.25% in PBS. After 2 h incubation, the medium was replaced by 100 μL DMSO (99% dimethyl sulfoxide, Sigma-Aldrich). Cell viability was then measured at wavelength of 560 nm in a Modulus® Microplate Photometer (Turner BioSystems Inc., Sunnyvale, CA, USA). Results were expressed as percentage of cell survival relative to untreated cells [2].

### 6.2.1. Cell Viability in Corneal Cells

The cytotoxicity of several concentrations of free APG, Loaded Lipid Nanoparticles, and the Lipid Nanoparticles (without APG) were evaluated on HCE-2 cells. The HCE-2 cell line was selected to analyse the compatibility of the formulations on corneal cells after topical administration.

Samples were incubated at different times to simulate real conditions of the contact between the formulation and the cornea in humans. For this reason, cell viability was tested during 5-, and 15-minutes incubation of the formulation. Pursuant to ISO 10993-5, percentages of cell viability above 80% are considered as non-cytotoxicity; within 80-60% weak; 60-40% moderate and below 40% strong cytotoxicity respectively [29].

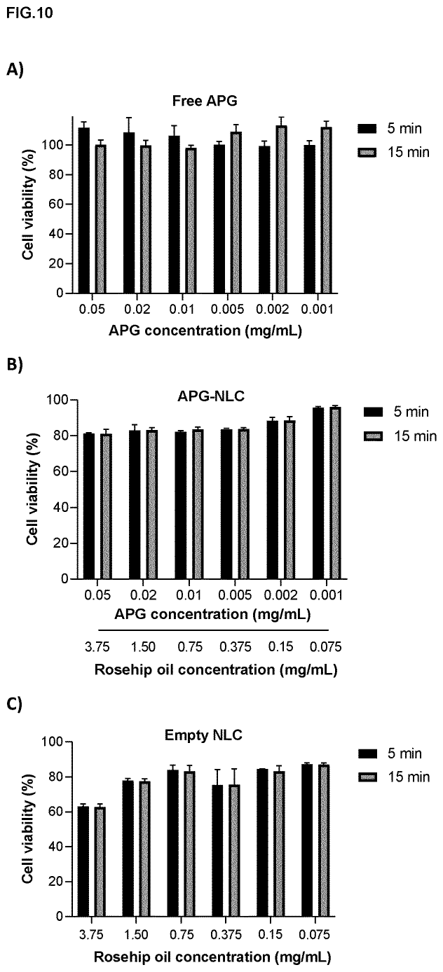

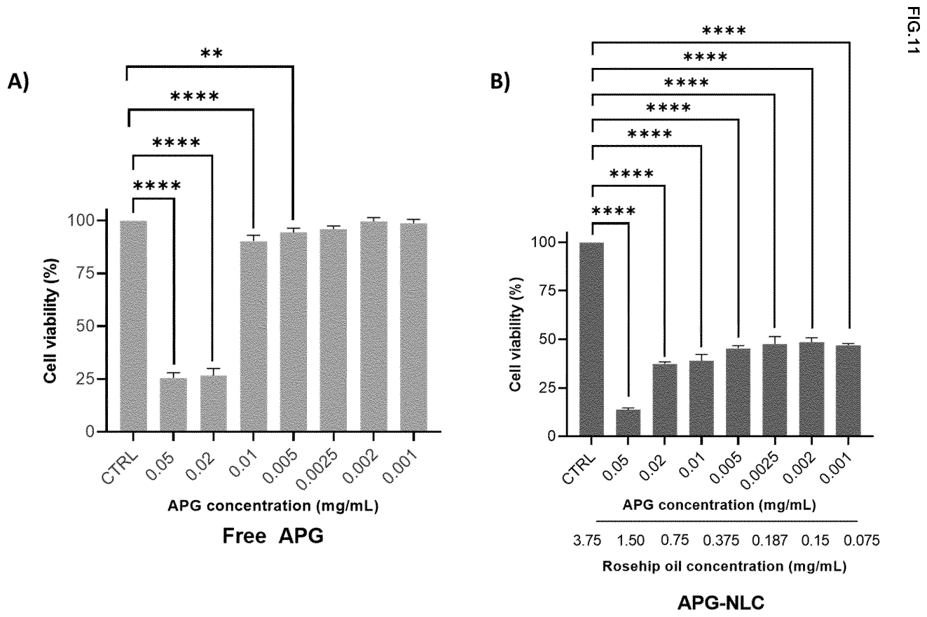

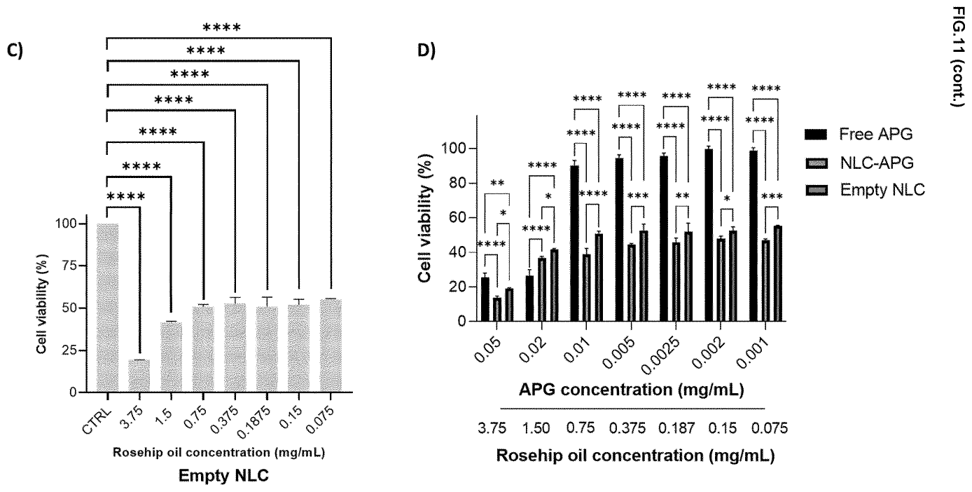

Results showed that after 5- and 15-minutes incubation, free APG did not cause relevant cytotoxic effects (≥80% viability). Free APG did not show toxicity at all the tested concentrations at all the time incubations (FIG. 10A). Loaded Lipid Nanoparticles after 5- and 15-minutes incubation showed a good cell viability in all the tested concentrations (FIG. 10B). Similarly, Lipid Nanoparticles resulted in a good cell viability for 5- and 15-minutes incubation from 0.02-0.001 mg/mL (FIG. 10C).

The most concentrated dilution showed a weak toxicity at both times. This effect could be produced for several reasons, such as its positive charge, which produce a higher electrostatic attraction to the negatively charged cell surfaces, leading to increases in oxidative stress and reactive oxygen species (ROS). Furthermore, the lipid NLCs has a high affinity for the cells, which facilitates the interaction between them. However, it can be observed that Loaded Lipid Nanoparticles were safer (higher cell viability) in all the tested concentrations. This effect could be related with the protective activity of APG on these cells [30].

### 6.2.2. Cytotoxic Capacity in Ocular Tumoral Cells

Furthermore, the inventors have studied the cytotoxic capacity of the composition of present invention in ocular tumoral cells. The study was performed in the cancer cell line UM 92.1 and showed that free APG caused significant toxicity from 0.005-0.05 mg/mL (FIG. 11A). Remarkably, Loaded Lipid Nanoparticles and Lipid Nanoparticles exerted a great cytotoxicity against the cancer cells in all the tested concentrations. The cytotoxic effect exerted on the tumoral cells was statically studied between the three different treatments (Table 1, FIG. 11D).

TABLE 4
Statistically significant differences between the treatments in UM 92.1 cell line.

| | 0.05 | 0.02 | 0.01 | 0.005 | 0.0025 | 0.002 | 0.001 |
|---|---|---|---|---|---|---|---|
| Free APG vs. | **** | **** | **** | **** | **** | **** | **** |
| NLC-APG | p < 0.0001 | p < 0.0001 | p < 0.0001 | p < 0.0001 | p < 0.0001 | p < 0.0001 | p < 0.0001 |
| Free APG vs. | ** | **** | **** | **** | **** | **** | **** |
| Empty NLC | p < 0.01 | p < 0.0001 | p < 0.0001 | p < 0.0001 | p < 0.0001 | p < 0.0001 | p < 0.0001 |
| NLC-APG vs. | * | * | **** | *** | ** | * | *** |
| Empty NLC | p < 0.05 | p < 0.05 | p < 0.0001 | p < 0.0005 | p < 0.001 | p < 0.05 | p < 0.0005 |

It can be observed that in all studied concentrations Loaded Lipid Nanoparticles showed a significantly higher antitumoral effect than free APG (p<0.0001), probably, as mentioned previously, because of the slow release of APG and the increased penetration of the NLCs into the cancer cells, leading to a higher cytotoxic effect. Lipid Nanoparticles also showed a significantly higher antitumoral effect than free APG in all concentrations probably due to the positive charge of the NLCs, which had been reported to increase the toxicity due to the high interaction between negatively charged surface of cells and the positive surface of NLCs. The cytotoxic effect of Loaded Lipid Nanoparticles and Lipid Nanoparticles was statistically significant. In all the tested concentrations, Loaded Lipid Nanoparticles resulted more cytotoxic in the UM cell line, which highlighted the antitumoral potential of APG when it was encapsulated into the NLCs [31,32].

### 6.3. Cellular Uptake

To evaluate the internalization of Loaded Lipid Nanoparticles in HCE-2 cells, 1×10⁵ cell·mL⁻¹ HCE-2 were grown in eight-well chamber slider (Ibidi®, Gräfelfing, Germany) until 80% confluence and posteriorly incubated with Loaded Lipid Nanoparticles with the fluorescent dye Nile Red (NR) at different times (5, 15 and 30 minutes) at 37° C. Non-internalized NPs were removed by washing three times with PBS and cells were fixed with 4% paraformaldehyde for 30 min at 25° C. Subsequently, cells were subjected to PBS washes and then, the nuclei were stained with 4′,6-diamidino-2-phenylindole (DAPI) for 10 min at 25° C. Then, cells were subjected to PBS washes, and Alexa Fluor™ 488 conjugated Wheat Germ Agglutinin (WGA) was used to stain cell membranes for 30 min at 25° C. Cells were washed and finally, mounting solution (PBS) was added for microscopic analysis. Images were acquired using a Leica Thunder Imager DMI8 (Leica Microsystems GmbH, Wetzlar, Germany) with a 63× oil immersion objective lens [33].

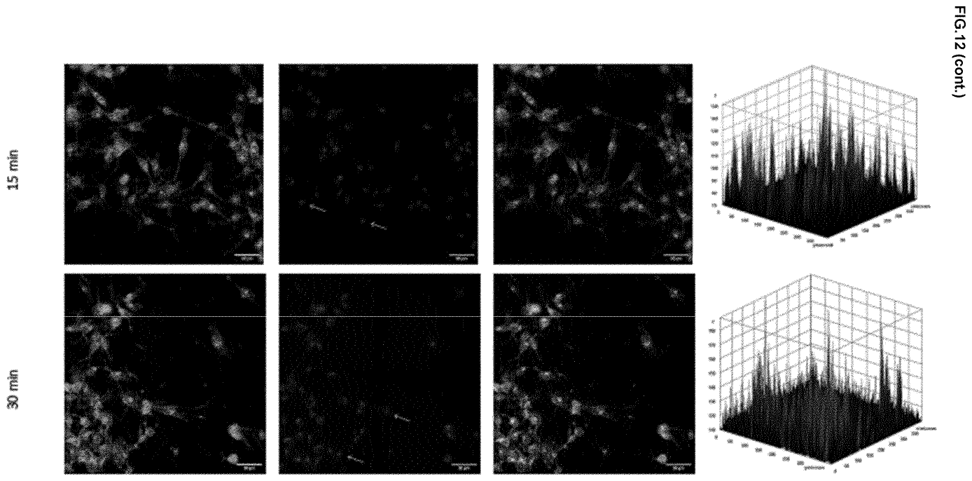

Cellular uptake of Loaded Lipid Nanoparticles was analysed in the HCE-2 cell line (FIG. 12).

After different incubation timepoints, the fluorescent NLCs were visualized by fluorescence microscopy. The nucleus was visualized with DAPI and cell membrane with Alexa Fluor™ 488-WGA to a better bio-localisation of NLCs. In the merged images, the Loaded Lipid Nanoparticles were visualized inside the cells, which meant that the particles were able to penetrate inside them without changing the morphology of the corneal cells. It has been reported that toxic substances could change the morphology of the cell membrane, such as benzalkonium chloride, which promotes vacuolization of the corneal cells. Moreover, the fluorescence signal had increased along with the incubation time. The analysis with Interactive 3D Surface Plot of ImageJ confirmed this fact and allowed to discern that 30 min incubation of Loaded Lipid Nanoparticles displayed higher intensity than cells incubated 5 or 15 min. Furthermore, no fluorescence was observed in the control cells.

### Example 7: Antiangiogenic Activity

To determine the antiangiogenic effects of Loaded Lipid Nanoparticles, a modified choriolantoic membrane (CAM) test was used. It was carried out using fertilised chicken eggs incubated at 37° C. and 85% humidity from the farm GALLSA (Tarragona, Spain). A lateral window was opened on the eggshell on the 3rd day of incubation and after 24 h of stabilization, 40 μL of the sample were inoculated to the CAM. Afterwards the membrane was sealed and incubated for 48 h. The controls of the experiments were NaCl as a normal angiogenic development, and basic fibroblast growth factor (bFGF) as a pro-angiogenic control (20 μL at 10 ng·mL⁻¹). Once CAM were evaluated, membranes were fixed by adding 4% paraformaldehyde overnight at 4° C. Next, membranes were extracted and observed using a binocular loupe. Afterwards, the obtained images were processed and the density of the vessels in the CAM was automatically measured using ImageJ vessel analysis plugin [34].

To study the antiangiogenic capacity of free APG, Loaded Lipid Nanoparticles, and Lipid Nanoparticles, an in vitro assessment using CAM of embryonated eggs was carried out. As shown in FIG. 13, Loaded Lipid Nanoparticles possessed a significantly lower (p<0.01) vascular density than the negative control (NaCl). Although free APG seems to exert antiangiogenic effects, these effects are not significant. Moreover, Lipid Nanoparticles did not seem to have any effect on the blood vasculature, being significantly higher in Loaded Lipid Nanoparticles.

### Example 8: Antibacterial Activity

Resazurin assay was used to quantify the metabolic activity, as it is proportional to the number of bacteria and their viability (n=5). Bacterial strain used was Staphylococcus aureus. The samples were washed twice with PBS and incubated with 300 μL of resazurin sodium salt at 30 μg/mL (Sigma-Aldrich, Spain) for 30 min at 37° C. The absorbance was measured at 570 and 600 nm using 100 μL of each sample (Infinite M Nano, TECAN, Switzerland) [35].

Antibacterial assessments after 4 h of incubation demonstrated the ability of both Loaded Lipid Nanoparticles as well as Lipid Nanoparticles to possess antibacterial metabolic capacity reduction (FIG. 14), significantly higher than the untreated control (p<0.005). This demonstrates the antibacterial potential against ocular infections of the formulations developed.

### Example 9: Anti-Inflammatory Activity In Vivo Assessments

In vivo anti-inflammatory effectiveness was carried out throughout the evaluation test for the inflammation prevention ability and the anti-inflammatory efficacy. Assays were carried out using New Zealand male albino rabbits (n=3/group), described previously. The activity of Loaded Lipid Nanoparticles in comparison with free APG, Lipid Nanoparticles, and NaCl 0.9% (control group) was measured. The inflammation prevention study consisted of the ocular application of 50 μL of each formulation. After 30 min of exposure, an inflammatory stimulus, 50 μL of 0.5% sodium arachidonate (SA) dissolved in PBS, was instilled in the right eye and the left eye was used as a control. In the anti-inflammatory treatment study, the inflammatory stimulus was applied 30 min before than the application of each formulation tested. The evaluation of prevention and treatment of each formulation were carried out from the first application up to 210 min, according to the Draize modified test scoring system [16].

In vivo anti-inflammatory efficacy was assayed to explore the capacity of the NLCs to prevent and treat ocular inflammation through two different tests. In addition, the in vivo inflammation treatment was assessed. Treatments were applied after 30 min of SA exposure, and the degree of inflammation was quantified. FIG. 15A revealed that the degree of inflammation was significantly reduced after the first 30 minutes post-administration of Loaded Lipid Nanoparticles. Free APG were able to treat fast the inflammation due to the NLCs had a controlled release. Moreover, Lipid Nanoparticles exerted anti-inflammatory activity after 90 minutes of instillation. Comparing the Loaded Lipid Nanoparticles with Lipid Nanoparticles, it can be observed that Loaded Lipid Nanoparticles had a significantly higher anti-inflammatory effects than Lipid Nanoparticles after 2 h after their application, probably due to APG prolonged release.

In vivo inflammatory prevention test showed significant differences between the degree of inflammation of APG formulations or physiological serum during all the timepoints tested (FIG. 15B). Nevertheless, eyes treated with Loaded Lipid Nanoparticles presented a faster swelling reduction rather than free APG, mainly owing to tear clearance in case of free APG and the improved ocular surface adherence of lipid nanoparticles, thus presenting longer residence time in the cornea. Loaded Lipid Nanoparticles exhibited significant differences regarding positive control over the time. Otherwise, Lipid Nanoparticles also showed an anti-inflammatory effect in vivo. Initially, the effect was similar to the Loaded Lipid Nanoparticles activity, but after 90 min of treatment it can be observed a significant anti-inflammatory effect produced by APG since significant differences between Lipid Nanoparticles and Loaded Lipid Nanoparticles were obtained [36]. Thus, Loaded Lipid Nanoparticles exhibited a preventive effect of inflammation caused by the sustained release of APG and the synergic activity of the vehicle, which could attribute to an initial anti-inflammatory effect. Hence, it can be concluded that the controlled release system based on Loaded Lipid Nanoparticles has ocular anti-inflammatory activity, both for prevention level and inflammation treatment. Furthermore, the vehicle showed anti-inflammatory effect, which could be attributed to the rosehip oil, that possesses that kind of activity

### Example 10: In Vivo Biodistribution

In vivo biodistribution assays were obtained by applying two 50 μL-administrations separated by 5 min of clearance of either Loaded Lipid Nanoparticles with NR or NR solution into the conjunctival sac of New Zealand albino rabbits, massaging the eye after each administration. After 3 h, the animals were sacrificed and the eyes were enucleated and transferred into paraformaldehyde 4% in PBS during 24 h, and then transferred to a solution formed by paraformaldehyde 4% and sucrose 30%. 24 h later, eyes were conserved in a O.C.T. compound cryostat embedding medium, and then freeze at −80° C. Afterwards, the freeze eyes were cut using a cryostat (Leica CM 3050 S, Leica Microsystems GmbH, Wetzlar, Germany) and the cellular nucleus were stained with DAPI. Fluorescence images were obtained using a Leica Thunder Imager DMI8 (Leica Microsystems GmbH, Wetzlar, Germany) and quantified using ImageJ software [37].

Loaded Lipid Nanoparticles was topically administered on New Zealand albino rabbits, to visualize the in vivo biodistribution of the sample (FIG. 16). After 3 h from the ophthalmic administration, the animals were sacrificed, the eyes were collected and subsequently cut into slices. Mean fluorescent intensity (MFI) of whole eye was measured by ImageJ software, and the results showed that MFI was practically the double in comparison to the control eye (MFI 1327.549±526.168 vs 756.442±268.446 respectively), which means that Loaded Lipid Nanoparticles after 3 h were located into the eye. Specifically, Loaded Lipid Nanoparticles were able to reach the posterior segment of the eye, being distributed mainly in the retina. Moreover, the smaller cuts of each tissue, showed that Loaded Lipid Nanoparticles had also a high interaction with the cornea and ciliary muscles, in which it can be observed that they were accumulated.

### REFERENCES

[1] Hantera M M. Trends in dry eye disease management worldwide. Clin Ophthalmol 2021; 15:173.
[2] López-Machado A, Diaz-Garrido N, Cano A, Espina M, Badia J, Baldomà L, et al. Development of lactoferrin-loaded liposomes for the management of dry eye disease and ocular inflammation. Pharmaceutics 2021; 13 (10):1698.
[3] Huang L, Gao H, Wang Z, Zhong Y, Hao L, Du Z. Combination nanotherapeutics for dry eye disease treatment in a rabbit model. Int J Nanomedicine 2021; 16:3631.
[4] Salehi B, Venditti A, Sharifi-Rad M, Kręgiel D, Sharifi-Rad J, Durazzo A, et al. The therapeutic potential of apigenin. Int J Mol Sci 2019; 20 (6):1305.
[5] Ali F, Rahul, Naz F, Jyoti S, Siddique Y H. Health functionality of apigenin: A review. Int J Food Prop 2016; 20 (6):1197-238.
[6] Clinical Trial to Evaluate the Reduction of Cardiovascular Risk—ClinicalTrials.gov n.d. https://clinicaltrials.gov/ct2/show/NCT04114916?term=apigenin&draw=4&rank=3 (accessed Nov. 19, 2021).
[7] Zick S M, Wright B D, Sen A, Arnedt J T. Preliminary examination of the efficacy and safety of a standardized chamomile extract for chronic primary insomnia: a randomized placebo-controlled pilot study. BMC Complement Altern Med 2011; 11:78.
[8] Apigenbell 60 cápsulas Jellybell|Naturitas n.d. https://www.naturitas.es/p/suplementos/fitoterapia/compuestos-herbarios/apigenbell-60-capsulas-jellybell?gclid=CjwKCAiAs92MBhAXEiwAXTi25z_Q5qx2aeNYvzbrUCyGRG6PfdA91wTK XvtWHr461rXXYtFP_cDcZxoCkrAQAvD_BwE (accessed Nov. 19, 2021).
[9] Apigenina 90 cápsulas de 50 mg Swanson I Naturitas n.d. https://www.naturitas.es/p/suplementos/suplementos-compuestos/apigenina-90-capsulas-de-50 mg-swanson (accessed Nov. 19, 2021).
[10] Optrex—colirio para el picor de ojos n.d. https://www.optrex.es/gama-optrex/colirio/optrex-colirio-calmante-para-el-picor-de-ojos/optrex-colirio-calmante-para-el-picor-de-ojos/(accessed Nov. 19, 2021).
[11] Optiben—Irritación ocular n.d. https://optiben.cinfa.com/Producto/7/alivio-natural-de-la-irritacin-ocular (accessed Nov. 19, 2021).
[12] Wang M, Firrman J, Liu L S, Yam K. A review on flavonoid apigenin: Dietary intake, ADME, antimicrobial effects, and interactions with human gut microbiota. Biomed Res Int 2019; 7010467.
[13] Fangueiro J F, Calpena A C, Clares B, Andreani T, Egea M A, Veiga F J, et al. Biopharmaceutical evaluation of epigallocatechin gallate-loaded cationic lipid nanoparticles (EGCG-LNs): In vivo, in vitro and ex vivo studies. Int J Pharm 2016; 502 (1-2):161-9.
[14] Agarwal P, Craig J P, Rupenthal I D. Formulation considerations for the management of dry eye disease. Pharmaceutics 2021; 13 (2):207.
[15] Sánchez-López E, Ettcheto M, Egea M A, Espina M, Cano A, Calpena A C, et al. Memantine loaded PLGA PEGylated nanoparticles for Alzheimer's disease: In vitro and in vivo characterization. J Nanobiotechnology 2018; 16 (1):1-16.
[16] Sánchez-López E, Esteruelas G, Ortiz A, Espina M, Prat J, Muñoz M, et al. Dexibuprofen biodegradable nanoparticles: one step closer towards a better ocular interaction study. Nanomaterials 2020; 10 (4).
[17] López-Machado A, Diaz N, Cano A, Espina M, Badia J, Baldomá L, et al. Development of topical eye-drops of lactoferrin-loaded biodegradable nanoparticles for the treatment of anterior segment inflammatory processes. Int J Pharm 2021; 609:121188.
[18] Esteruelas G, Halbaut L, Garcia-Torra V, Espina M, Cano A, Ettcheto M, et al. Development and optimization of Riluzole-loaded biodegradable nanoparticles incorporated in a mucoadhesive in situ gel for the posterior eye segment. Int J Pharm 2022; 612:121379.
[19] Alshehri S M, Shakeel F, Ibrahim M A, Elzayat E M, Altamimi M, Mohsin K, et al. Dissolution and bioavailability improvement of bioactive apigenin using solid dispersions prepared by different techniques. Saudi Pharm J 2019; 27 (2):264-73. [20] Stability of Dispersions|3P Instruments n.d. https://www.3p-instruments.com/measurement-methods/stability-turbiscan/(accessed Nov. 22, 2021).
[21] Liu P, De Wulf O, Laru J, Heikkila T, Van Veen B, Kiesvaara J, et al. Dissolution studies of poorly soluble drug nanosuspensions in non-sink conditions. AAPS Pharm Sci Tech 2013; 14 (2):748.
[22] Huang D, Chen Y S, Rupenthal I D. Hyaluronic acid coated albumin nanoparticles for targeted peptide delivery to the retina. Mol Pharm 2017; 14 (2):533-45.
[23] Lagarto A, Vega R, Guerra I, González R. In vitro quantitative determination of ophthalmic irritancy by the chorioallantoic membrane test with trypan blue staining as alternative to eye irritation test. Toxicol In Vitro 2006; 20 (5):699-702.
[24] Xiong C, Chen D, Liu J, Liu B, Li N, Zhou Y, et al. A rabbit dry eye model induced by topical medication of a preservative benzalkonium chloride. Invest Ophthalmol Vis Sci 2008; 49 (5):1850-6.
[25] Holzchuh R, Villa Albers M B, Osaki T H, Igami T Z, Santo R M, Kara-Jose N, et al. Two-year outcome of partial lacrimal punctal occlusion in the management of dry eye related to Sjögren syndrome. Curr Eye Res 2011; 36 (6):507-12.
[26] Lin T K, Zhong L, Santiago J L. Anti-inflammatory and skin barrier repair effects of topical application of some plant oils. Int J Mol Sci 2017; 19 (1):70.
[27] Kiralan M, Yildirim G. Rosehip (Rosa canina L.) Oil. Fruit Oils Chem Funct 2019:803-14.
[28] Barbaraci C, Giurdanella G, Leotta C G, Longo A, Amata E, Dichiara M, et al. Haloperidol metabolite I I valproate ester (S)-(−)-MRJF22: preliminary studies as a potential multifunctional agent against uveal melanoma. J Med Chem 2021; 64 (18):13622-32.
[29] López-Garcia J, Lehocký M, Humpoliček P, Sáha P. HaCaT keratinocytes response on antimicrobial atelocollagen substrates: extent of cytotoxicity, cell viability and proliferation. J Funct Biomater 2014; 5 (2):57.
[30] Yang W, Wang L, Mettenbrink E M, Deangelis P L, Wilhelm S. Nanoparticle toxicology. Annu Rev Pharmacol Toxicol 2021; 61:269-89.
[31] Cagle P, Idassi O, Carpenter J, Minor R, Goktepe I, Martin P. Effect of rosehip (Rosa canina) extracts on human brain tumor cell proliferation and apoptosis. J Cancer Ther 2012; 2012 (05):534-45.
[32] Mármol I, Jimdnez-Moreno N, Ancin-Azpilicueta C, Osada J, Cerrada E, Rodriguez-Yoldi M J. A combination of Rosa Canina extracts and gold complex favors apoptosis of Caco-2 cells by increasing oxidative stress and mitochondrial dysfunction. Antioxidants 2019; 9 (1):17.
[33] Gonzalez-Pizarro R, Parrotta G, Vera R, Sánchez-López E, Galindo R, Kjeldsen F, et al. Ocular penetration of fluorometholone-loaded PEG-PLGA nanoparticles functionalized with cell-penetrating peptides. Nanomedicine 2019; 14 (23):3089-104.
[34] Esteruelas G, Souto E B, Espina M, Garcia M L, Świtalska M, Wietrzyk J, et al. Diclofenac loaded biodegradable nanoparticles as antitumoral and antiangiogenic therapy. Pharmaceutics 2022; 15 (1):102.
[35] Elmsmari F, Maria Delgado L, Duran-Sindreu F, Perez R A, Luisa Garcia M, Teulé Trull M, et al. Novel strategies enhancing endodontic disinfection: antibacterial biodegradable calcium hydroxide nanoparticles in an ex vivo model. Int J Pharm 2023; 648 (November):123627.
[36] Strugata P, Gładkowski W, Kucharska A Z, Sokół-Łetowska A, Gabrielska J. Antioxidant activity and anti-inflammatory effect of fruit extracts from blackcurrant, chokeberry, hawthorn, and rosehip, and their mixture with linseed oil on a model lipid membrane. Eur J Lipid Sci Technol 2016; 118 (3):461-74.
[37] Swetledge S, Carter R, Stout R, Astete C E, Jung J P, Sabliov C M. Stability and ocular biodistribution of topically administered PLGA nanoparticles. Sci Rep 2021; 11(1):1-11.

1. A composition comprising lipid nanoparticles, said lipid nanoparticles comprising a) rosehip oil;
b) at least one solid lipid;
c) at least one surfactant;
d) optionally, at least one active ingredient;
wherein the composition is suitable for ocular topical administration.

2. The composition of any one of the preceding claims, wherein the solid lipid is selected from monoglycerides, diglycerides, triglycerides, cholesterols, steroids, fatty alcohols, glycerol esters glyceryl tridecanoate, glycerol trilaurate, glyceryl trimyristate, glyceryl tripalmitate, glyceryl tristearate, Hydrogenated coco-glycerides, Hard fat types, mixtures of triglycerides and/or diglycerides and/or monoglycerides and/or glycerol, acyl glycerols, glyceryl monostearate, glyceryl distearate, glyceryl monooleate, glyceryl dibehenate, glyceryl palmitostearate, waxes, cetyl palmitate, fatty acids, stearic acid, palmitic acid, decanoic acid, behenic acid, glycerol stearate citrate, polyethylene glycol monostearate, cyclodextrin para-acyl-calix-arenes, or mixtures thereof, preferably glyceryl dibehenate.

3. The composition of claim 1 or 2, wherein the at least one surfactant is selected from a cationic surfactant, a non-cationic surfactant, or a combination thereof.

4. The composition of any one of the preceding claims, wherein the cationic surfactant is selected from the group consisting of dimethyldioctadecylammonium bromide (DDAB), dioleoyl phosphatidylethanolamine, 1,2-distearyloxy-N,N-dimethyl-3-aminopropane, 1,2 dioleyl-oxy-N,N-dimethyl-3-aminopropane, 1,2-dilinoleyloxy-N,N-dimethyl-3-aminopro-pane, 1,2-dilinolenyloxy-N,N-dimethyl-3-aminopropane, cetyltri-methylammonium bromide, 3ß-[N(N′,N′-dimethylaminoethane)-carbamoyl]cholesterol, 1,2-dioleoyl-3-trimethylammonium-propane, 1,2-dimyristoyl-3-trimethylammonium-propane, 1,2-stearoyl-3-tri-methylammonium-propane, N-(4-carboxybenzyl)-N,N-dimethyl-2,3-bis(oleoyloxy) propan-1-aminium, 1-Palmitoyl-2-oleoyl-sn-glycero-3-phosphoethanol-amine, N,N-di-(P-stearoylethyl)-N,N-dimethyl-ammonium chloride, benzalkonium chloride, cetylpyridinium chloride, cetrimide, N-[1-(2,3-dioleoyloxy)propyl]-N,N,N-trimethylammonium chloride, 1,2-dilineoyl-3-dimethylammonium-propane, 1,2-dilinoleyloxy-3-N,N-dimethylaminopropane, 1,2-dilinoleyloxy-keto-N,N-dimethyl-3-aminopropane, 1,2-dilinoleyl-4-(2-dimethylaminoethyl)-[1,3]-dioxolane(3-o-[2″-(meth oxypolyethyleneglycol 2000) succinoyl]-1,2-dimyristoyl-sn-glycol, R-3-[(ω-methoxy-poly(ethyleneglycol)2000) carbamoyl]-1,2-dimyristyloxlpropyl-3-amine, cetyltrimethyl-ammonium bromide, octadecylamine, 1-oleoyl-rac-glycerol, octadecyl quaternized carboxymethyl chitosan, hexadecyl trimethyl ammonium bromide, preferably wherein the cationic surfactant is dimethyl-dioctadecyl-ammonium bromide (DDAB).

5. The composition of any one of the preceding claims, wherein the non-cationic surfactant is selected from the group consisting of polysorbate 80, soya lecithin, sodium dodecyl sulphate, polysorbate 20, polysorbate 40, polysorbate 60, PEG-30 glyceryl stearate, cholic acid, phosphatidyl choline, phospholipids with phosphatidycholine, egg lecithin, poloxamer 188, poloxamer 407, poloxamer 184, poloxamer 338, poloxamine 908, tyloxopol, taurocholate sodium salt, taurodeoxycholicacid sodium salt, sodium glycocholate, sodium oleate, cholesteryl hemisuccinate, butanol, sodium cholate, nonionic polyoxyethylene, non-ionic amphiphilic surfactant with an alkyl moiety and an ethylene oxide chain, palmitic acid, stearic acid, mixtures of palmitic and stearic acid, lecithin, polyglycerol 6-distearate, caprylyl/capryl glucoside, coco-glucoside, sucrose palmitate, sucrose stearate, sucrose distearate, tyloxapol, lecithin, cetylpyridinium chloride, sorbitan laurate, polyethylene glycol ether of cetyl or stearyl alcohol, castor oil polyoxyethylene ether, macrogolglycerol ricinoleate, dioctyl sodium sulfosuccinate, monooctylphosphoric acid sodium, hexadecyl trimethyl ammonium bromide, polyvinyl alcohol, polyoxyethylene (40) stearate, polyethylene glycol-polypropylene glycol-polyethylene glycol triblock copolymer, olyoxyethylene nonylphenyl ether, hexadecyltrimethylammonium bromide, sodium dodecyl sulfate, sodium cholate, stearate sodium hydrolysed polyvinyl alcohol 9000-10000 MW, dioctyl sulfosuccinate, taurocholate, 4-dodecylbenzenesulfonic acid, long chain carboxylic acid, alkyldiphenyloxide disulfonate, or mixtures thereof, preferably polysorbate 80.

6. The composition of any one of the preceding claims, wherein the at least one active ingredient is encapsulated in the lipid nanoparticles.

7. The composition of any one of the preceding claims, wherein the at least one active ingredient is apigenin.

8. The composition of any one of the preceding claims, wherein said lipid nanoparticles further comprise a coating, preferably wherein said coating comprises hyaluronic acid.

9. The composition of any one of the preceding claims, wherein said lipid nanoparticles comprise
a. 0.01-30% (w/v) rosehip oil
b. 1.0-50% (w/v) solid lipid, preferably glyceryl dibehenate;
c. 0.001-10% (w/v) cationic surfactant, preferably DDAB;
d. optionally 0.00-10% (w/v) non-cationic surfactant, preferably polysorbate 80;
e. optionally 0.00-10% (w/v) active ingredient, preferably apigenin.

10. The composition of any one of the preceding claims, wherein said lipid nanoparticles comprise
a. 2.5-12.5% (w/v) rosehip oil
b. 1.0-30% (w/v) solid lipid, preferably glyceryl dibehenate;
c. 0.025-0.5% (w/v) cationic surfactant, preferably DDAB;
d. 1.0-5.0% (w/v) non-cationic surfactant, preferably polysorbate 80;
e. optionally 0.1-2.5% (w/v) active ingredient, preferably apigenin.

11. The composition of any one of the preceding claims, wherein said lipid nanoparticles comprise 1.0-6.5% (w/v) solid lipid and 0.03-0.4% (w/v) cationic surfactant.

12. The composition of any one of the preceding claims, wherein said lipid nanoparticles comprise
a. 2.5-10% (w/v) rosehip oil
b. 1.0-6.5% (w/v) solid lipid, preferably glyceryl dibehenate;
c. 0.03-0.4% (w/v) cationic surfactant, preferably DDAB;
d. 3.0-5.0% (w/v) non-cationic surfactant, preferably polysorbate 80;
e. optionally 0.1-2.5% (w/v) active ingredient, preferably apigenin.

13. The composition of any one of the preceding claims, wherein said lipid nanoparticles comprise
a. 3.0% (w/v) rosehip oil,
b. 4.5% (w/v) glyceryl dibehenate,
c. 0.05% (w/v) DDAB,
d. 3.5% (w/v) polysorbate 80,
e. optionally 0.1% (w/v) apigenin.

14. The composition of any one of the preceding claims, wherein the lipid nanoparticles further comprise from 0.00001%-0.001% (w/v), preferably 0.0001% (w/v) hyaluronic acid, more preferably wherein said hyaluronic acid coats the lipid nanoparticles.

15. A composition comprising lipid nanoparticles according to any one of the preceding claims for use in the treatment, amelioration or prevention of ocular diseases, such as ocular inflammation, glaucoma, ocular tumors, bacterial and viral infections of the eye, age-related macular degeneration, cataracts, diabetic retinopathy, or Dry Eye Disease (DED).
